(19) United States
(12) Patent Application Publication
(10) Pub. No.: US 2021/0406771 A1
(43) Pub. Date: Dec. 30, 2021

(54) SYSTEMS AND METHODS FOR USING ARTIFICIAL INTELLIGENCE TO EVALUATE LEAD DEVELOPMENT

(71) Applicant: CATAILYST INC., Needham, MA (US)

(72) Inventor: Arindrajit Basak, Needham, MA (US)

(21) Appl. No.: 17/359,203

(22) Filed: Jun. 25, 2021

Related U.S. Application Data
(60) Provisional application No. 63/044,734, filed on Jun. 26, 2020.

(51) Int. Cl. G06N 20/00; G06F 40/205; G16H 70/40; G16H 10/40

(52) U.S. Cl. CPC G06N 20/00; G16H 10/40; G16H 70/40; G06F 40/205

(57) ABSTRACT

Systems and methods for providing a computer system for evaluating a candidate subject are provided. A program with instructions to receive a first communication amongst various communications is provided. Each communication has text data and the received communication is associated with a candidate subject. The program has instructions to extract a plurality of information from the text data of the received communication. A tag is assigned to each of the information in a subset of information. A subset of tags is applied in which an evaluation of the candidate subject is obtained.

### CROSS-REFERENCE TO RELATED APPLICATION

The present application claims priority to U.S. Provisional Patent Application No. 63/044,734, entitled “Systems and Methods for Using Artificial Intelligence to Evaluate Lead Development,” filed Jun. 26, 2020, which is hereby incorporated by reference in its entirety.

### TECHNICAL FIELD

The present disclosure relates to systems and methods for providing a computer system for evaluating a candidate subject (e.g., for lead development).

### BACKGROUND

Consider that biotechnological and pharmaceutical companies are under public eye when developing and releasing a new drug onto the market. In addition to the company and product being regulated at every phase by the Food and Drug Administration (FDA), in order to get public recognition for discovery, the companies often hold news releases, perform conference presentations, and submit SEC filings. Therefore, a significant portion of the information relating to products in development for biotechnological and pharmaceutical companies are available to the public. For instance, a drug discovery process often begins at a university or at a research lab. The findings of this process often become either published research articles, or patents. However, the ability to turn the research conducted at these facilities into a viable drug is challenging. These ventures are extremely costly and have a low probability of success. Additionally, gaining information related to the product is difficult to procure as the information is expensive, sold amongst several database companies and experiences a lag in time that it takes to be processed and published, lacks in comprehensiveness of information, and does not have integrated data. Therefore, subjects are searching for ways to increase their likelihood for success by choosing the best products for investment.

Prior solutions have attempted to solve this problem by aggregating and summaries publications related to a particular topic. While these prior solutions have successfully summarized a wealth of information, these solutions have yet to provide a mechanism that can extract additional information related to these topics in a time efficient manner. Furthermore, these prior solutions cannot provide meaningful evaluations and analysis from the wealth of information based on their aggregated summaries.

Thus, prior to the present disclosure there existed a need for a better information database that can be used to advise subjects in making better evaluations and decisions.

The information disclosed in this Background of the Invention is only for enhancement of understanding of the general background of the invention and should not be taken as an acknowledgement or any form of suggestion that this information forms the prior art already known to a person skilled in the art.

### SUMMARY

Given the above background, what is needed in the art are systems and methods for evaluating a lead associated with a candidate subject.

Accordingly, various aspects of the present disclosure are directed to providing systems and methods for information gathering, categorization, and, optionally, evaluation.

As aspect of the present disclosure is directed to providing systems and methods for providing an evaluation of candidate subject evaluations that is based on a categorization of information associated with the candidate subject. The systems and methods of the present disclosure allow for receiving a plurality of communications, with each communication in the plurality of communications including a respective plurality of text data, which facts pertaining to a candidate subject. In some embodiments, each communication in the plurality of communications in a published communication, which allows for the systems and methods of the present disclosure to maintain accurate, precise, and relevant data pertaining to the candidate subject. Using a trained classifier, the systems and methods of the present disclosure extract a corresponding plurality of information from the respective text data of a first communication in the plurality of communications. In some embodiments, the corresponding plurality of information is extracted ipsissimis verbis from the first communication. In some embodiments, the corresponding plurality of information extracted from the first communication conveys the facts of the first communication in a different form. In some embodiments, the trained classifier extracts the corresponding plurality of information from the first communication by evaluating a plurality of sentences and then evaluating a corresponding paragraph that includes a respective sentence in the plurality of sentences. In some embodiments, the trained classifier extracts the corresponding plurality of information from the first communication by evaluating a plurality of paragraphs and then evaluating a corresponding sentence that is included in a respective paragraph in the plurality of paragraphs. Accordingly, the trained classifier is capable of extracting information from the first communication in a variety of ways dependent on the candidate subject, a characteristic of the first communication (e.g., a form of the first communication, such as a scholarly article form or a financial report form), a type of information (e.g., a computational equation, a numerical value, a word, a string of characters, etc.) or and the like. From this, the trained classifier and a reference database are used to assign a tag to each respective information in a subject of information of the corresponding plurality of information extracted from the first communication. By assigning the tag, the systems and methods of the present disclosure allow for categorization (e.g., classification) of information into one or more bins. In doing so, the systems and methods of the present disclosure reduce a computation burden by retaining essential information of the first communication that is pertinent to a respective tag without having to retain unnecessary information found in the first communication. Furthermore, the tag enables the systems and methods of the present disclosure to conduct an evaluation of the candidate subject that considers information from the plurality of communications, which allows for a robust and comprehensive output.

Accordingly, an aspect of the present description relates to systems and methods for providing a computer system for evaluating a candidate subject. The computer system includes a program with instructions to receive a first communication amongst various communications. In some embodiments, the program includes instructions for polling for the first communication based on an association with the candidate subject. Each communication includes text data. Moreover, the first communication is associated with the candidate subject. The program includes instructions to extract a plurality of information from the text data of the first communication. A tag is assigned to each respective information in a subset of information of a corresponding plurality of information of the first communication. A subset of tags is applied in. From this, an evaluation of the candidate subject is obtained.

The evaluating of the present disclosure provides a user with an ability to gain an insight into various candidate subjects (e.g., one or more companies, one or more products, etc.) by obtaining public communications relating to the candidate subject. The evaluation is conducted with reference to one or more tags that is assigned and that represent a desired characteristic to the user (e.g., associated with a candidate subject). The present disclosure provides improved systems and methods for providing a dynamically updated database that includes information compiled from one or more publicly available sources. The information retained by the database is extracted from information relating to the candidate subject from a corpus of communications, such as product data and financial data (e.g., stock information and market data). A trained classifier is provided to extract, assign, and distribute the information from public resources.

In more detail, one aspect of the present disclosure is directed to providing a computer system for evaluating a candidate subject. The computer system includes at least one processor, and a memory storing at least one program for execution by the at least one processor. The at least one program includes instructions for receiving, in electronic form, a first communication in a plurality of communications. Each communication in the plurality of communications includes a respective plurality of text data. Moreover, the first communication is associated with the candidate subject. The at least one program further includes instructions for extracting, using a trained classifier, a corresponding plurality of information from the respective text data of the first communication. The instructions also include assigning a tag to each respective information in a subset of information of the corresponding plurality of information using the trained classifier and a reference database. In this way, the at least one program collectively assigns a first plurality of tags in a set of tags to the corresponding plurality of information. Additionally, the at least one program includes instructions for applying, to the trained classifier and the reference database, a subset of tags of the first plurality of tags. Accordingly, an evaluation of the candidate subject is obtained.

In some embodiments, the candidate subject includes an entity, a tangible asset, an intangible asset, or a combination thereof.

In some embodiments, the receiving is conducted in response to a request to evaluate the candidate subject.

In some embodiments, prior to the receiving, the at least one program further includes instructions for polling for the first communication based on the association with the candidate subject, and in accordance with a determination that the first communication exists, conducting the receiving.

In some embodiments, the applying is conducted in response to a request to evaluate the candidate subject.

In some embodiments, the request to evaluate the candidate subject is provided by a remote device.

In some embodiments, the request to evaluate the candidate subject is provided on a recurring basis.

In some embodiments, the reference database includes a corpus of communications. Prior to the receiving, the at least one program further includes instructions for training the trained classifier to evaluate the communication based on the corpus of communications.

In some embodiments, the corpus of communications is associated with the candidate subject. In some embodiments, the corpus of communications is uniquely associated with the candidate subject.

In some embodiments, the at least one program includes instructions for adding the first communication to the corpus of communications.

In some embodiments, the corpus of communications includes the corresponding plurality of information of the first communication, the first plurality of tags of the first communication, or both.

In some embodiments, the text data of the first communication includes unstructured text data. Additionally, the receiving further includes parsing the unstructured text data for use with the trained classifier.

In some embodiments, the first communication is received from a predetermined remote source.

In some embodiments, the first communication is received from a first source.

Accordingly, prior to the extracting, the at least one program includes instructions for validating the first source.

In some embodiments, the validating the first source includes determining a type of source associated with the first source.

In some embodiments, in accordance with a determination of the type of source associated with the first source, the validating the first source further includes receiving a validation of the first source from a human subject.

In some embodiments, in accordance with a determination of the type of source associated with the first source, the validating the first source further includes assigning a weight of credibility to the first communication.

In some embodiments, the type of source includes a press media, a news media, a filing with an entity, a release from the entity, or a combination thereof.

In some embodiments, the corresponding plurality of information of the extracting contains a portion, less than all, of the text data.

In some embodiments, the trained classifier conducts the extracting in accordance with a corresponding plurality of heuristic instructions that is associated with the extracting.

In some embodiments, the corresponding plurality of heuristic instructions includes a first subset of heuristic instructions that extracts the first plurality of text data of the first communication into a first subset of information that contains a portion, less than all, of the corresponding plurality of information. Furthermore, the corresponding plurality of heuristic instructions includes a second subset of heuristic instructions that extracts a second plurality of text data of the second communication into a second subset of information that contains a portion, less than all, of the corresponding plurality of information.

In some embodiments, the first subset of information and the second subset of information are disjoint subsets of the corresponding plurality of information.

In some embodiments, the at least one program further includes instructions for conducting the extracting in accordance with the first plurality of heuristic instructions and the assigning based on the first subset of information. As such, in accordance with a determination based on the assigning of the first subset of information, the at least one program further includes instructions for conducting the extracting in accordance with the second plurality of heuristic instructions and the assigning based on the second subset of information.

In some embodiments, the set of tags includes a subset of tier tags. As such, the first subset of information is assigned a first tier tag in the subset of tier tags. The second subset of information is associated with a second tier tag in the subset of tier tags. Moreover, the second tier tag is lower than the first tier tag in the plurality of tier tags.

In some embodiments, the set of tags includes a subset of category tags. The assigning includes a respective category tag in the subset of category tags to the corresponding of information.

In some embodiments, the subset of category tags includes a plurality of primary category tags. Each primary category tag in the plurality of primary category tags includes a corresponding plurality of secondary category tags in the subset of category tags. The assigning further includes, in accordance with a determination of a respective category tag in the subset of category tags to the corresponding of information, a secondary category tag.

In some embodiments, the plurality of primary category tags includes an analyst report tag, an annual report tag, an asset acquisition tag, an asset sale tag, a clinical development update tag, a corporate update tag, a discard not relevant tag, a financing tag, an individual tag, a change in roles tag, a license agreement tag, a market research report tag, an entity merger tag, an entity acquisition tag, a new entity tag, an opinion tag, an option agreement tag, an “other” tag, a partnership tag, a preclinical update tag, a quarterly report tag, a regulatory report tag, a scientific analysis tag, a scientific publication tag, a patent publication tag, a future event tag, or a combination thereof.

In some embodiments, the at least one program further include instructions for conducting the receiving, the extracting, the assigning, and the applying for a second communication in the plurality of communications. As such, the at least one program further includes instructions for forming the subset of tags of the first plurality of tags based on an evaluation of the first plurality of tags of the corresponding information of the first communication with the second plurality of the corresponding information of the second communication.

In some embodiments, the evaluation formed by the applying includes a prediction of a future event, a prediction of a future communication in the plurality of communications, a comparison of the candidate subject to a second subject, or a combination thereof.

Another aspect of the present disclosure is directed to providing a method of evaluating a candidate subject at a computer system. The computer system includes one or more processors, and memory coupled to the one or more processors, the memory including one or more programs configured to be executed by the one or more processors. As such, the method includes receiving, in electronic form, a first communication in a plurality of communications. Each communication in the plurality of communications includes a respective plurality of text data. Moreover, the first communication is associated with the candidate subject. The method includes extracting, using a trained classifier, a corresponding plurality of information from the respective text data of the first communication. In addition, the method includes assigning, using the trained classifier and a reference database, a tag to each respective information in a subset of information of the corresponding plurality of information. From this, a first plurality of tags in a set of tags is collectively assigned to the corresponding plurality of information. Furthermore, the method includes applying, to the trained classifier and the reference database, a subset of tags of the first plurality of tags. In this way, the method obtains an evaluation of the candidate subject.

Yet another aspect of the present disclosure is directed to providing a non-transitory computer readable storage medium. The non-transitory computer readable storage medium stores instructions, which when executed by a computer system, cause the computer system to perform a method. The method includes receiving, in electronic form, a first communication in a plurality of communications. Each communication in the plurality of communications includes a respective plurality of text data. Moreover, the first communication is associated with the candidate subject. The method includes extracting, using a trained classifier, a corresponding plurality of information from the respective text data of the first communication. In addition, the method includes assigning, using the trained classifier and a reference database, a tag to each respective information in a subset of information of the corresponding plurality of information. From this, a first plurality of tags in a set of tags is collectively assigned to the corresponding plurality of information. Furthermore, the method includes applying, to the trained classifier and the reference database, a subset of tags of the first plurality of tags. In this way, the method obtains an evaluation of the candidate subject.

Other features and advantages of the invention will be apparent from, or are set forth in more detail in, the accompanying drawings, which are incorporated herein, and the following Detailed Description, which together serve to explain certain principles of exemplary embodiments of the present invention.

### BRIEF DESCRIPTION OF THE DRAWINGS

FIG. 1 illustrates an exemplary system topology including a classification system and one or more client devices, in accordance with an embodiment of the present disclosure;

FIG. 2 illustrates various modules and/or components of a classification system, in accordance with an embodiment of the present disclosure;

FIG. 3 illustrates various modules and/or components of a client device, in accordance with an embodiment of the present disclosure;

FIGS. 4A, 4B, 4C, 4D, 4E, and 4F collectively provide a flow chart of methods for evaluating a lead development associated with a candidate subject, in which dashed boxes represent optional elements in the flow chart, in accordance with an embodiment of the present disclosure;

FIG. 5 illustrates a user interface for presenting a listing of a plurality of communication; in accordance with an embodiment of the present disclosure;

FIG. 6 illustrates another user interface for presenting a corresponding plurality of information extracted from a respective communication, in accordance with an embodiment of the present disclosure; and

FIG. 7 illustrates yet another user interface for presenting a corresponding plurality of information extracted from a respective communication, in accordance with an embodiment of the present disclosure.

In the figures, reference numbers refer to the same or equivalent parts of the present invention throughout the several figures of the drawing.

### DETAILED DESCRIPTION

The present description relates to systems and methods for evaluating a lead development associated with a candidate subject. Specifically, the systems and methods include receiving a first communication in a plurality of communications. Each communication includes a plurality of text data. Furthermore, the first communication is associated with a candidate subject. By receiving the first communication, the systems and methods of the present disclosure reduce a burden on a subject by omitting a requirement that the subject inputs the communication. The systems and methods include extracting a corresponding plurality of information from the respective text data of the first communication. The extracting, the systems and methods can assign, using the trained classifier and a reference database, a tag to each respective information in a subset of information of the corresponding plurality of information, thereby collectively assigning a first plurality of tags in a set of tags to the corresponding plurality of information.

Reference will now be made in detail to various embodiments of the present invention(s), examples of which are illustrated in the accompanying drawings and described below. While the invention(s) will be described in conjunction with exemplary embodiments, it will be understood that the present description is not intended to limit the invention(s) to those exemplary embodiments. On the contrary, the invention(s) is/are intended to cover not only the exemplary embodiments, but also various alternatives, modifications, equivalents and other embodiments, which may be included within the spirit and scope of the invention as defined by the appended claims.

It will also be understood that, although the terms first, second, etc. may be used herein to describe various elements, these elements should not be limited by these terms. These terms are only used to distinguish one element from another. For instance, a first candidate subject could be termed a second candidate subject, and, similarly, a second candidate subject could be termed a first candidate subject, without departing from the scope of the present disclosure. The first candidate subject and the candidate subject are both candidate subjects, but they are not the same candidate subject.

The terminology used in the present disclosure is for the purpose of describing particular embodiments only and is not intended to be limiting of the invention. As used in the description of the invention and the appended claims, the singular forms “a,” “an,” and “the” are intended to include the plural forms as well, unless the context clearly indicates otherwise. It will also be understood that the term “and/or” as used herein refers to and encompasses any and all possible combinations of one or more of the associated listed items. It will be further understood that the terms “includes” and/or “including,” when used in this specification, specify the presence of stated features, integers, steps, operations, elements, and/or components, but do not preclude the presence or addition of one or more other features, integers, steps, operations, elements, components, and/or groups thereof.

The foregoing description included example systems, methods, techniques, instruction sequences, and computing machine program products that embody illustrative implementations. For purposes of explanation, numerous specific details are set forth in order to provide an understanding of various implementations of the inventive subject matter. It will be evident, however, to those skilled in the art that implementations of the inventive subject matter may be practiced without these specific details. In general, well-known instruction instances, protocols, structures and techniques have not been shown in detail.

The foregoing description, for purpose of explanation, has been described with reference to specific implementations. However, the illustrative discussions below are not intended to be exhaustive or to limit the implementations to the precise forms disclosed. Many modifications and variations are possible in view of the above teachings. The implementations are chosen and described in order to best explain the principles and their practical applications, to thereby enable others skilled in the art to best utilize the implementations and various implementations with various modifications as are suited to the particular use contemplated.

In the interest of clarity, not all of the routine features of the implementations described herein are shown and described. It will be appreciated that, in the development of any such actual implementation, numerous implementation-specific decisions are made in order to achieve the designer's specific goals, such as compliance with use case- and business-related constraints, and that these specific goals will vary from one implementation to another and from one designer to another. Moreover, it will be appreciated that such a design effort might be complex and time-consuming, but nevertheless be a routine undertaking of engineering for those of ordering skill in the art having the benefit of the present disclosure.

As used herein, the term “if” may be construed to mean “when” or “upon” or “in response to determining” or “in response to detecting,” depending on the context. Similarly, the phrase “if it is determined” or “if [a stated condition or event] is detected” may be construed to mean “upon determining” or “in response to determining” or “upon detecting [the stated condition or event]” or “in response to detecting [the stated condition or event],” depending on the context.

As used herein, the term “about” or “approximately” can mean within an acceptable error range for the particular value as determined by one of ordinary skill in the art, which can depend in part on how the value is measured or determined, e.g., the limitations of the measurement system. For example, “about” can mean within 1 or more than 1 standard deviation, per the practice in the art. “About” can mean a range of ±20%, ±10%, ±5%, or ±1% of a given value. Where particular values are described in the application and claims, unless otherwise stated, the term “about” means within an acceptable error range for the particular value. The term “about” can have the meaning as commonly understood by one of ordinary skill in the art. The term “about” can refer to ±10%. The term “about” can refer to ±5%.

As used herein, the term “dynamically” means an ability to update a program while the program is currently running.

Furthermore, as used herein, the term “classifier” and “trained classifier” are used interchangeably herein unless expressly stated otherwise.

Moreover, as used herein, the term “parameter” refers to any coefficient or, similarly, any value of an internal or external element (e.g., a weight and/or a hyperparameter) in an algorithm, model, regressor, and/or classifier that can affect (e.g., modify, tailor, and/or adjust) one or more inputs, outputs, and/or functions in the algorithm, model, regressor and/or classifier. For example, in some embodiments, a parameter refers to any coefficient, weight, and/or hyperparameter that can be used to control, modify, tailor, and/or adjust the behavior, learning and/or performance of an algorithm, model, regressor, and/or classifier. In some instances, a parameter is used to increase or decrease the influence of an input (e.g., a feature) to an algorithm, model, regressor, and/or classifier. As a nonlimiting example, in some instances, a parameter is used to increase or decrease the influence of a node (e.g., of a neural network), where the node includes one or more activation functions. Assignment of parameters to specific inputs, outputs, and/or functions is not limited to any one paradigm for a given algorithm, model, regressor, and/or classifier but can be used in any suitable an algorithm, model, regressor, and/or classifier architecture for a desired performance. In some embodiments, a parameter has a fixed value. In some embodiments, a value of a parameter is manually and/or automatically adjustable. In some embodiments, a value of a parameter is modified by a validation and/or training process for an algorithm, model, regressor, and/or classifier (e.g., by error minimization and/or backpropagation methods, as described elsewhere herein). In some embodiments, an algorithm, model, regressor, and/or classifier of the present disclosure comprises a plurality of parameters. In some embodiments the plurality of parameters is n parameters, where: n≥2; n≥5; n≥10; n≥25; n≥40; n≥50; n≥75; n≥100; n≥125; n≥150; n≥200; n≥225; n≥250; n≥350; n≥500; n≥600; n≥750; n≥1,000; n≥2,000; n≥4,000; n≥5,000; n≥7,500; n≥10,000; n≥20,000; n≥40,000; n≥75,000; n≥100,000; n≥200,000; n≥500,000, n≥1×10⁶, n≥5×10⁶, or n≥1×10⁷. In some embodiments n is between 10,000 and 1×10⁷, between 100,000 and 5×10⁶, or between 500,000 and 1×10⁶.

Additionally, the terms “client,” “subject,” and “user” are used interchangeably herein unless expressly stated otherwise.

Furthermore, when a reference number is given an “i^{th}” denotation, the reference number refers to a generic component, set, or embodiment. For instance, a communication termed “communication i” refers to the i^{th} communication in a plurality of communications (e.g., a first communication 240-1 in a plurality of communications 240).

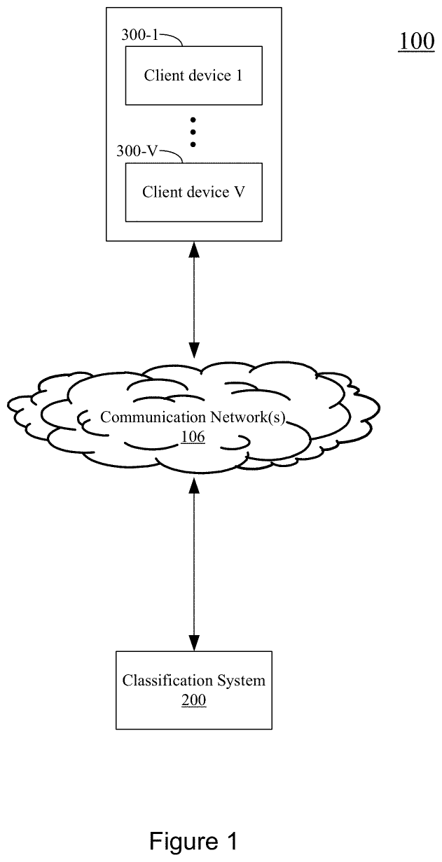

In the present disclosure, unless expressly stated otherwise, descriptions of devices and systems will include implementations of one or more computers. For instance, and for purposes of illustration in FIG. 1, a client device 300 is represented as single device that includes all the functionality of the client device 300. However, the present disclosure is not limited thereto. For instance, the functionality of the client device 300 may be spread across any number of networked computers and/or reside on each of several networked computers and/or by hosted on one or more virtual machines and/or containers at a remote location accessible across a communications network (e.g., communications network 106). One of skill in the art will appreciate that a wide array of different computer topologies is possible for the client device 300, and other devices and systems of the preset disclosure, and that all such topologies are within the scope of the present disclosure.

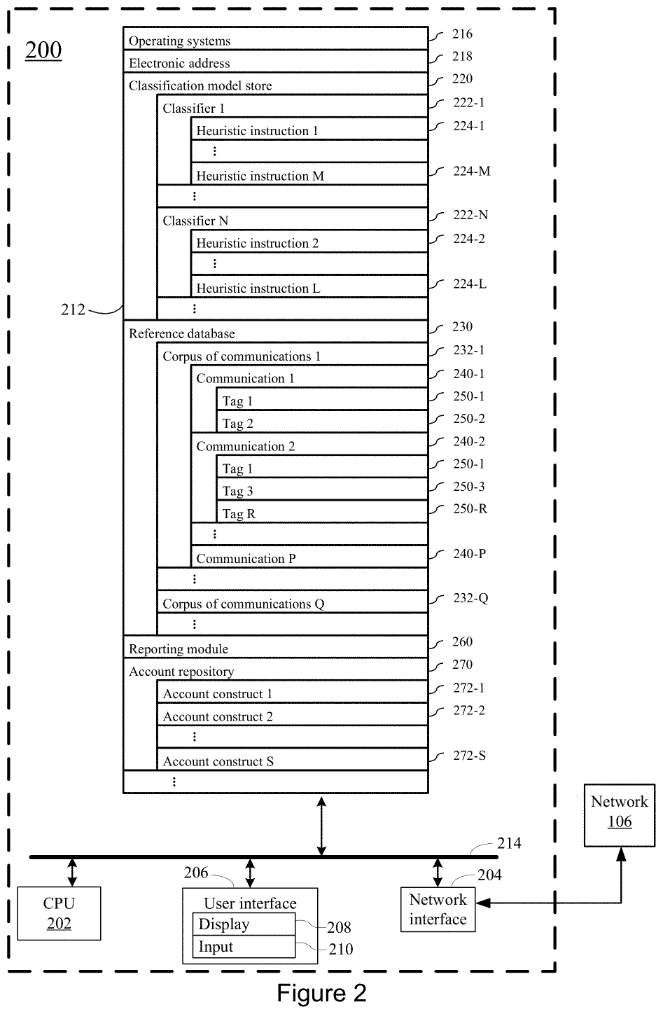

FIG. 1 illustrates an exemplary topology of an evaluation system 100 (e.g., a distributed-client system), which allows for evaluating a lead development associated with a candidate subject. The system 100 includes a classification system (e.g., classification system 200 of FIG. 2) that receives a communication (e.g., first communication 240-1 of FIG. 2). In some embodiments, the classification systems 200 receives the communication 240 by way of a communication network (e.g., communication network(s) 106 of FIG. 1). The system 100 includes one or more client devices 300 (e.g., computing devices) that provides a request for an evaluation of a candidate subject and/or receive the evaluation of the candidate subject from the system. In some embodiments, such a request is provided by way of the communications network 106.

A detailed description of a system 100 for evaluating a lead development associated with a candidate subject in accordance with the systems and methods of the present disclosure is described in conjunction with FIG. 1 through FIG. 3. As such, FIG. 1 through FIG. 3 collectively illustrate an exemplary topology of the system 100 in accordance embodiments of the present disclosure.

More particularly, in the topology, there is a classification system 200 for receiving one or more communications and/or evaluating a lead development associated with a candidate subject based on the one or more communications. The classification system 200 utilizes one or more trained classifiers (e.g., classifiers 222 of FIG. 2) and/or a reference database (e.g., reference database 230 of FIG. 2) to ascertain a characteristic of the one or more communication 240. For instance, in some embodiments, the trained classifier 222 extracts a plurality of information from a respective communication and one or more tags (e.g., tags 250 of FIG. 2) to the plurality of information.

Referring to FIG. 1, the classification system 200 is configured receive one or more communications 240 and provide an evaluation of a candidate subject based on the one or more communications 240. In some embodiments, the classification system 200 receives the one or more communications 240 from a client device 300 and/or a remote device, such as a remote database and/or a remote server associated with the system 100. In this way, the communication 240 is provided in electronic form to the classification system 200 (e.g., in an electronic unformatted structured format, in an electronic structured format, or a combination thereof) by transmission within the communication network 106.

In some embodiments, the classification system 200 receives a communication 240 wirelessly through radio-frequency (RF) signals. In some embodiments, such signals are in accordance with an 802.11 (Wi-Fi), Bluetooth, or ZigBee standard.

In some embodiments, the classification system 200 is not proximate to a subject and/or does not have wireless capabilities or such wireless capabilities are not used for the purpose of receiving a communication 240 and/or a request for an evaluation of a candidate subject. In such embodiments, a communication network 106 is utilized to receive a communication from a source (e.g., client device 300) to the classification system 200.

Examples of networks 106 include, but are not limited to, the World Wide Web (WWW), an intranet and/or a wireless network, such as a cellular telephone network, a wireless local area network (LAN) and/or a metropolitan area network (MAN), and other devices by wireless communication. The wireless communication optionally uses any of a plurality of communications standards, protocols and technologies, including but not limited to Global System for Mobile Communications (GSM), Enhanced Data GSM Environment (EDGE), high-speed downlink packet access (HSDPA), high-speed uplink packet access (HSUPA), Evolution, Data-Only (EV-DO), HSPA, HSPA+, Dual-Cell HSPA (DC-HSPDA), long term evolution (LTE), near field communication (NFC), wideband code division multiple access (W-CDMA), code division multiple access (CDMA), time division multiple access (TDMA), Bluetooth, Wireless Fidelity (Wi-Fi) (e.g., IEEE 802.11a, IEEE 802.11ac, IEEE 802.11ax, IEEE 802.11b, IEEE 802.11g and/or IEEE 802.11n), voice over Internet Protocol (VoIP), Wi-MAX, a protocol for e-mail (e.g., Internet message access protocol (IMAP) and/or post office protocol (POP)), instant messaging (e.g., extensible messaging and presence protocol (XMPP), Session Initiation Protocol for Instant Messaging and Presence Leveraging Extensions (SIMPLE), Instant Messaging and Presence Service (IMPS)), and/or Short Message Service (SMS), or any other suitable communication protocol, including communication protocols not yet developed as of the filing date of the present disclosure.

In some embodiments, the classification system 200 receives a communication 240 directly from a respective source (e.g., directly from a client device 300 that generated the communication 240). In some embodiments, the classification system 200 receives a communication 240 from a remote device, such as an auxiliary server (e.g., from a remote application host server). In such embodiments, the auxiliary server is in communication with a client device 300 and receives one or more communications 240 from the client device 300. Accordingly, the auxiliary server provides the communication 240 to classification system 200. In some embodiments, the auxiliary server provides (e.g., polls for) one or more communications 240 on a recurring basis (e.g., each minute, each hour, each day, as specified by the auxiliary server and/or a user, etc.). However, the present disclosure is not limited thereto.

Of course, other topologies of the system 100 other than the one depicted in FIG. 1 are possible. For instance, in some embodiments, rather than relying on a communications network 106, the one or more client devices 300 wirelessly transmit information directly to the classification system 200. Further, in some embodiments, the classification system 200 constitutes a portable electronic device, a server computer, or in fact constitutes several computers that are linked together in a network or be a virtual machine and/or a container in a cloud-computing context. As such, the exemplary topology shown in FIG. 1 merely serves to describe the features of an embodiment of the present disclosure in a manner that will be readily understood to one of skill in the art.

Turning to FIG. 2 with the foregoing in mind, in some embodiments, the classification system 200 includes one or more computers. For purposes of illustration in FIG. 2, the classification system 200 is represented as a single computer that includes all of the functionality for evaluating a lead development associated with a candidate subject. However, the present disclosure is not limited thereto. In some embodiments, the functionality for providing a classification system 200 is spread across any number of networked computers, and/or resides on each of several networked computers, and/or is hosted on one or more virtual machines and/or one or more containers at a remote location accessible across the communications network 106. One of skill in the art will appreciate that any of a wide array of different computer topologies are used for the application and all such topologies are within the scope of the present disclosure.

An exemplary classification system 200 for evaluating a lead development associated with a candidate subject based on one or more communications 240 is provided. The classification system 200 includes one or more processing units (CPU's) 202, a network or other communications interface 204, a memory 212 (e.g., random access memory), and one or more communication busses 214 for interconnecting the aforementioned components. In some embodiments, the classification system 200 includes a user interface 206, the user interface 206 including a display 208 and an input 210 (e.g., keyboard, keypad, touch screen, etc.). In some embodiments, the memory 212 includes mass storage that is remotely located with respect to the central processing unit(s) 202. In other words, some data stored in the memory 212 may in fact be hosted on computers that are external to the classification system 200, but that can be electronically accessed by the classification system 200 over an Internet, intranet, or other form of network or electronic cable (illustrated as element 106 in FIG. 2) using network interface 204.

In some embodiments, the memory 212 of the classification system 200 for evaluating a lead development associated with a candidate subject based on one or more communications 240 stores:
an operating system 216 that includes procedures for handling various basic system services;
an electronic address 218 that is associated with the classification system 200;
a classification model store 220 that stores one or more classifiers 222, each classifier 222 including a corresponding plurality of heuristic instructions 224;
a reference database 230 that stores one or more corpus of communications 232, each corpus of communications 232 including a plurality of communications 240 and one or more tags 250 that is associated with a respective communication 240 in the plurality of communications 240;
a reporting module 260 for providing an evaluation of a candidate subject based on the one or more communications 240; and
an account repository 270 for retaining a plurality of account constructs 272, each account construct 272 corresponding to an account held with the classification system by a subject.

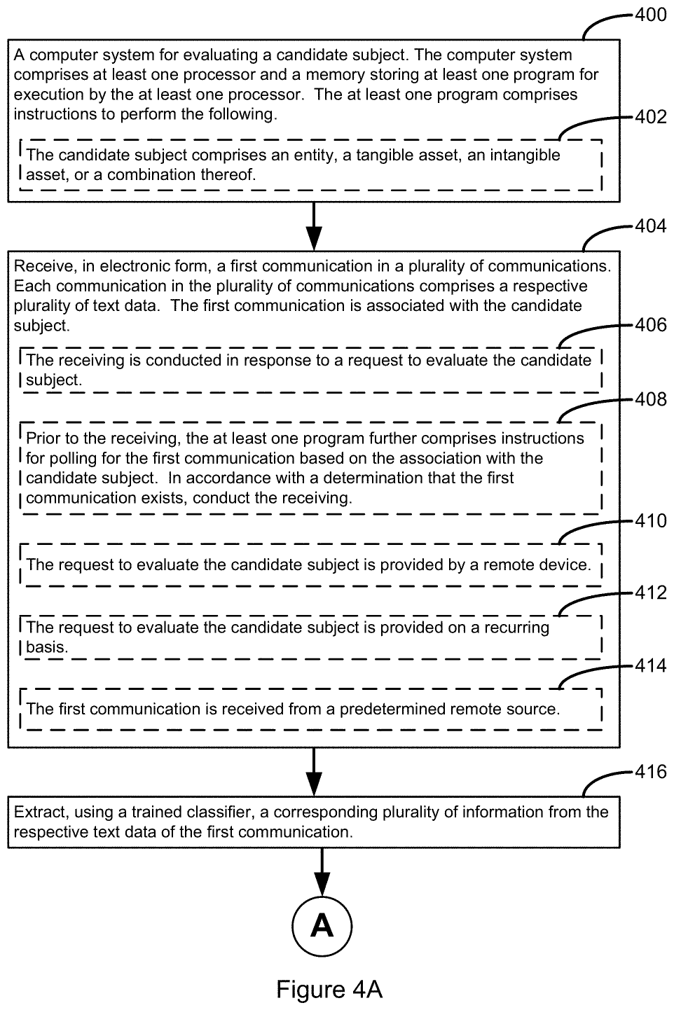

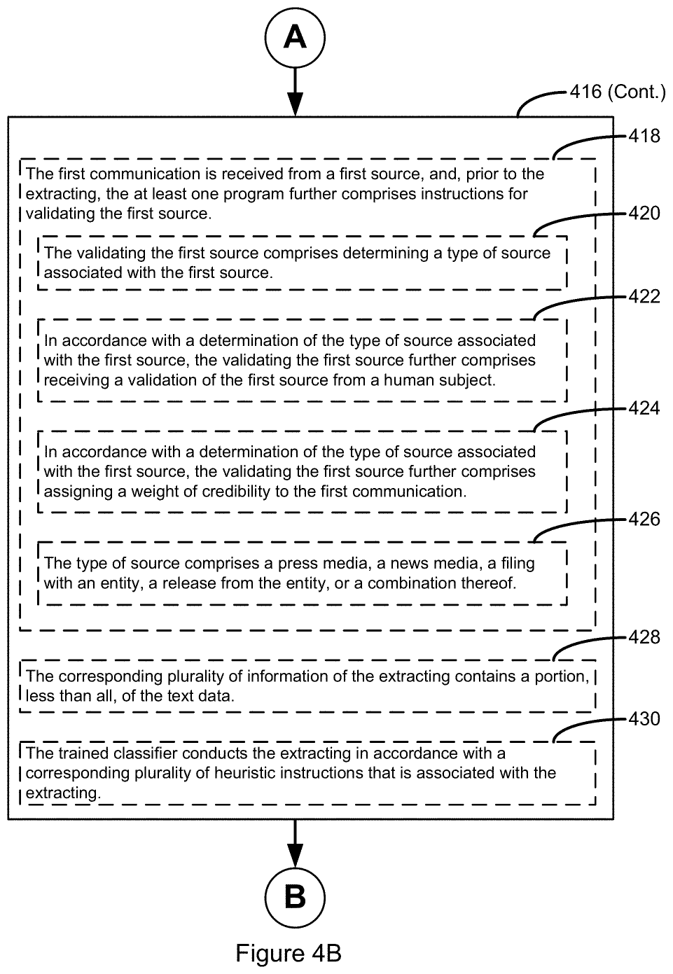

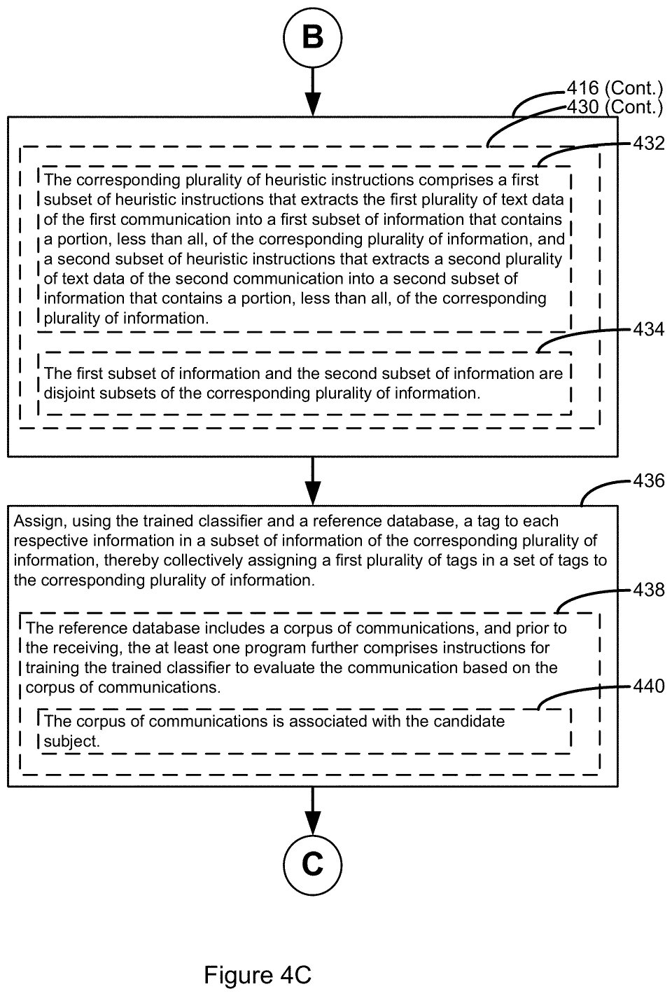

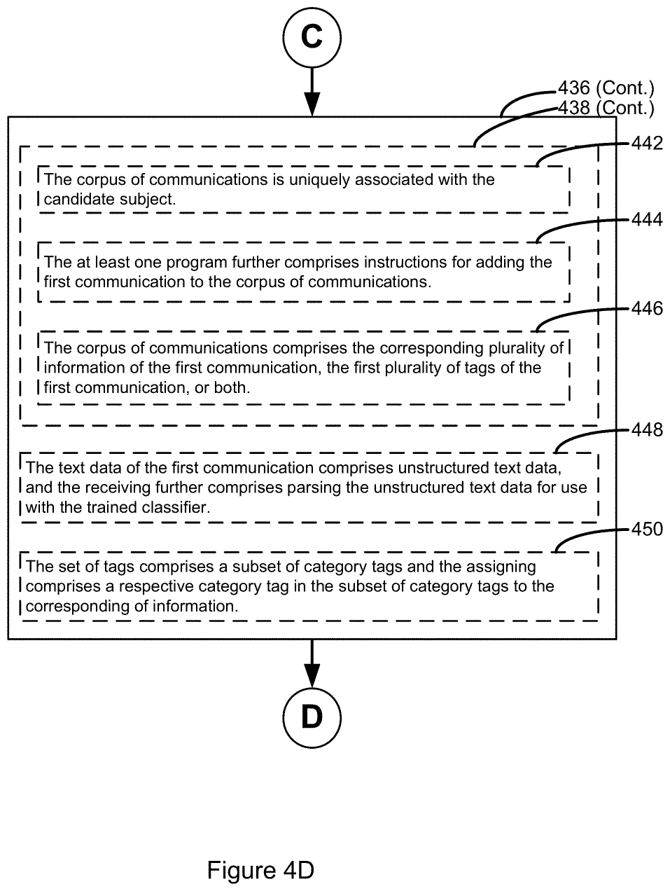

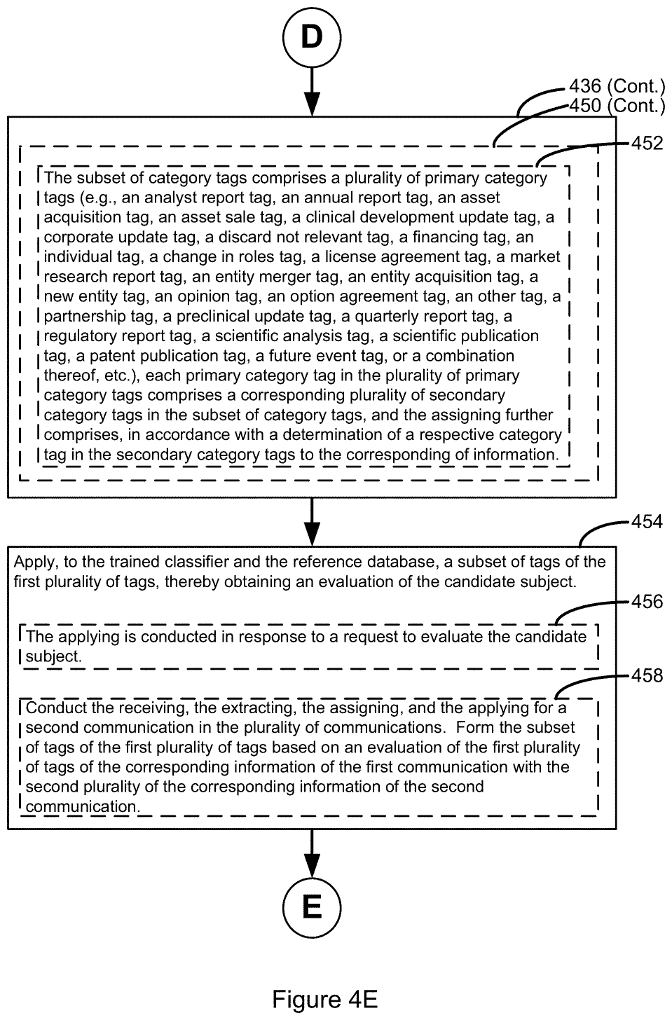

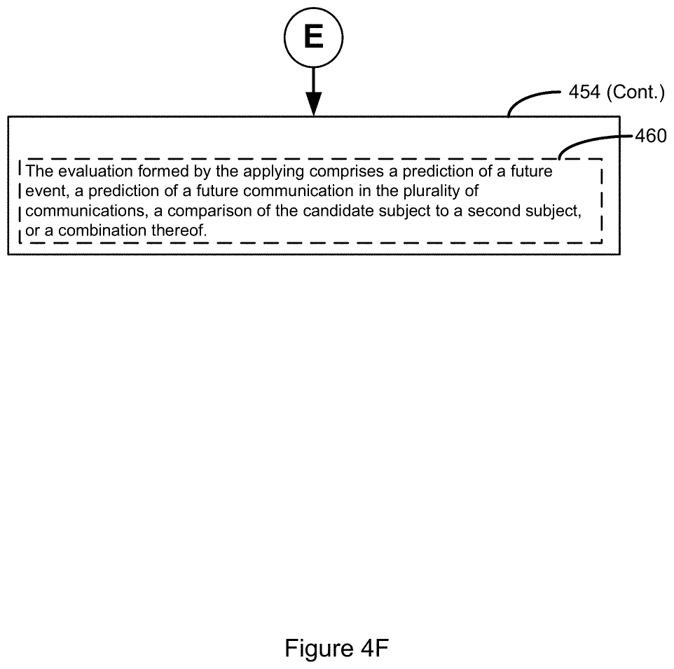

A classification model store 220 stores one or more classifiers 222 that facilitates extracting a plurality of information from a communication 240 (e.g., block 416 of FIG. 4A) and/or forming an evaluation of a candidate subject from the plurality of information extracted from one or more communications 240. In this way, in some embodiments, a respective classifier 222 in the one or more classifiers 222 extracts the plurality of information from the respective communication 240 in accordance with a plurality of heuristic instructions 224 (e.g., first heuristic instruction 224-1, second heuristic instruction 224-2, . . . , heuristic instruction M 224-M of FIG. 2). In some embodiments, the respective classifier 222 obtains an evaluation of the candidate subject for a subject based on the extracted plurality of information. By way of example, in some embodiments, a first classifier 222-1 is configured to extract a first plurality of information in accordance with at least a first heuristic instruction 224-1. Moreover, a second classifier 224-1 is trained on at least the first plurality of information that is extracted by at least the first classifier 222-1. In this way, the second classifier acts as a trained classifier 222. However, the present disclosure is not limited.

Accordingly, in some embodiments, due to the inherent complexity of understanding the underlying context of a communication 240, one classifier 222 is not capable of solving all natural language processing (NLP) problems when extracting the plurality of information from the communication 240. Moreover, one approach using a respective classifier 222 to solving a particular NLP problem is not always optimal for every NLP problem. Accordingly, the classification model store 220 stores a plurality of classifiers 222, which provides a more robust evaluation of the candidate subject.

In some embodiments, the classifier 222 is implemented as an artificial intelligence engine and may include gradient boosting models, random forest models, neural networks (NN), regression models, Naive Bayes models, and/or machine learning algorithms (MLA). In some embodiments, a MLA or a NN is trained from a training data set (e.g., corpus of communications 232 of FIG. 2) that includes one or more features identified through the extracted from a first communication 240-1. MLAs include supervised algorithms (such as algorithms where the features/classifications in the data set are annotated) using linear regression, logistic regression, decision trees, classification and regression trees, naïve Bayes, nearest neighbor clustering; unsupervised algorithms (such as algorithms where no features/classification in the data set are annotated) using Apriori, means clustering, principal component analysis, random forest, adaptive boosting; and semi-supervised algorithms (such as algorithms where an incomplete number of features/classifications in the data set are annotated) using generative approach (such as a mixture of Gaussian distributions, mixture of multinomial distributions, hidden Markov models), low density separation, graph-based approaches (such as mincut, harmonic function, manifold regularization), heuristic approaches, or support vector machines.

NNs include conditional random fields, convolutional neural networks, attention based neural networks, deep learning, long short term memory networks, or other neural models where the training data set includes a plurality of tumor samples, RNA expression data for each sample, and pathology reports covering imaging data for each sample.

While MLA and neural networks identify distinct approaches to machine learning, the terms may be used interchangeably herein. Thus, a mention of MLA may include a corresponding NN or a mention of NN may include a corresponding MLA unless explicitly stated otherwise. Training may include providing optimized datasets, labeling these traits as they occur in patient records, and training the MLA to predict or classify based on new inputs. Artificial NNs are efficient computing models which have shown their strengths in solving hard problems in artificial intelligence. They have also been shown to be universal approximators, that is, they can represent a wide variety of functions when given appropriate parameters.

Accordingly, in some embodiments a first classifier 222-1 is a neural network classification model, a second classifier 222-2 is a Naïve Bayes classification model, and the like. Furthermore, in some embodiments, the classifier 222 of the classification model store 220 includes decision tree classifiers (e.g., third classifier 222-3), a neural network classifier (e.g., fourth classifier 222-4), a support vector machine (SVM) classifier (e.g., fifth classifier 222-5), and the like. Moreover, in some embodiments, the classifier 222 used in the methods (e.g., method 400 of FIGS. 4A through 4F) described herein is a logistic regression algorithm, a neural network algorithm, a convolutional neural network algorithm, a support vector machine (SVM) algorithm, a Naive Bayes algorithm, a nearest neighbor algorithm, a boosted trees algorithm, a random forest algorithm, a decision tree algorithm, a clustering algorithm, or a combination thereof.

One of skill in the art will readily appreciate other classification models for use as a classifier 222 that is applicable to the systems and methods of the present disclosure. In some embodiments, the systems and methods of the present disclosure utilize more than one classifiers 222 to provide an evaluation of a candidate subject with an increased accuracy when extracting information from a communication 240 and/or obtaining the evaluation of the candidate subject. For instance, in some embodiments, each respective classifier 222 arrives at a corresponding evaluation when extracting information from a respective communication 240 and/or obtaining an evaluation of a candidate subject. Accordingly, the independently arrived extracted information from the communication 240 and/or the evaluation of the candidate subject of each respective classifier 222 is collectively verified through a comparison or amalgamation of the classifiers 222. From this, a cumulative extraction of information from the communication 240 and/or evaluation of the candidate subject is provided by the classification system 200.

Each classifier 222 includes a plurality of heuristic instructions 224 that describe one or more processes for the classifier 222 to follow (e.g., first classifier 222-1 of FIG. 2 includes a first plurality of heuristic instructions 224 including a first heuristic instruction 224-1 and a heuristic instruction M 224-M, second classifier 222-2 of FIG. 2 includes a second plurality of heuristic instructions 224-2 including a second heuristic instruction 224-2 and a heuristic instruction L 224-4, etc.). Each respective heuristic instruction 222 in the plurality of heuristic instructions 224 defines a framework for handling one or more parameters and/or decisions involved in extracting a plurality of information from a communication 240 and/or providing an evaluation from the extracted plurality of information. For instance, in some embodiments, a respective heuristic instruction 224 is formed from one or more feature vector, whereby each respective feature vector in the one or more feature vectors describes a positive and/or negative application of the heuristic instruction 224. By way of example, in some embodiments, the first classifier 224-1 is a decision tree classification model. Each node of a respective decision tree generated by the first classifier 224-1 represents a decision associated with a respective heuristic instruction 224 in the first plurality of heuristic instructions 224-1 of the first classifier 222-1. However, the present disclosure is not limited thereto.

In some embodiments, the plurality of heuristic instructions 224 utilizes historical results (e.g., provided by a human user), such as if a particular word has ever been associated with one or more tags 250. In some embodiments, the historical result is a simple historical result, which considers only the communications 240 in a predetermined period of time (within two years, within a day, etc.). In some embodiments, the historical result is a total historical result, that measures an average across all periods of time. In some embodiments, the historical result is a weighted history, that assigned a weighted average (e.g., more important to recent periods of time).

In some embodiments, the plurality of heuristic instructions 224 utilize one or more grammar inferences, such as by forming one or more relationships between clusters of words and/or synonyms to address natural language semantics. In some embodiments, the plurality of heuristic instructions 224 utilize parts-of-speech identifying mechanisms, such as identifying a string of characters as a noun, a verb, a quantity, etc. In some embodiments, the plurality of heuristic instructions 224 utilize a term frequency-inverse document frequency (TF-IDF), which determines a term frequency in a corpus of communications 232 or a communication 240. In some embodiments, this term frequency is normalized by a total number of terms in the corpus of communications 232 or the communication 240. In some embodiments, the plurality of heuristic instructions 224 this normalized term frequency is utilized to produce a rarity of a term, which is defined by a function of a total number of communications 240 in the corpus of communications 232 with the number of communications 240 that contain the term in the corpus of communications 232. However, the present disclosure is not limited thereto. Additional details and information regarding a plurality of heuristic instructions 224 of a respective classifier 222 can be found at Hemmati et al., 2018, “Investigating NLP-based Approaches for Predicting Manual Test Case Failure,” IEEE 11^{th} International Conference on Software Testing, Verification and Validation, pg. 309; Monsifrot et al., 2002, “A Machine Learning Approach to Automatic Production of Compiler Heuristics,” International Conference on Artificial Intelligence, Methodology, Systems, and Applications, pg. 41, each of which is hereby incorporated by reference in its entirety.

In some embodiments, a respective classifier 222 is an inter-pattern distance based classification model that includes a multi-layer network of threshold logic units (TLU), which provide a framework for pattern (e.g., characteristic) classification. This framework includes a potential to account for various factors including parallelism of data, fault tolerance of data, and noise tolerance of data. Furthermore, this framework provides representational and computational efficiency over disjunctive normal form (DNF) expressions and a classifier that is a decision tree classification model. In some embodiments, a TLU implements an (N−1) dimensional hyperplane partitioning an N-dimensional Euclidean pattern space into two regions. In some embodiments, one TLU neural network sufficiently classifies patterns in two classes if the two patterns are linearly separable. Compared to other classifiers 22, such as a classifier 222 that is a constructive learning classification models, the inter-pattern distance based classification model uses a variant TLU (e.g., a spherical threshold unit) as hidden neurons. Additionally, the distance based classification model determines an inter-pattern distance between each pair of patterns in a training data set (e.g., corpus of communications 232 of FIG. 2), and determines the weight values for the hidden neurons. This approach differs from other classification models that utilize an iterative classification process to determine the weights and thresholds for evaluating and providing a characteristic of a communication.

In some embodiments, a respective classifier 222 is a distance based classification model that utilizes one or more types of distance metric to determine an inter-pattern distance between each pair of patterns. For instance, in some embodiments, the distance metric is based on those described in Duda et al., 1973, “Pattern Classification and Scene Analysis,” Wiley, Print., and/or that described in Salton et al., 1983, “Introduction to Modern Information Retrieval,” McGraw-Hill Book Co., Print, each of which is hereby incorporated by reference in their entirety. Table 1 provides various types of distance metrics of the distance based classification model of the respective classifier 222.

TABLE 1
Exemplary distance metrics for the distance based classification model of
the respective classifier 222. Consider X^{p} = [X₁^{p}, . . . , Xₙ^{p}] and X^{q} = [X₁^{p}, . . . , Xₙ^{q}] to be two
pattern vectors. Also consider maxᵢ and minᵢ to be the maximum value and the minimum
value of an i^{th} attribute of the patterns in a data set (e.g., a text object and/or a text string),
respectively. The distance between X^{p} and X^{q} is defined as follows for each distance metric:

| Type | Distance Metric |
|---|---|
| Euclidean | $d ( {X}^{p} , {X}^{q} ) = \sqrt{{\sum }_{i = 1}^{n} {( {X}_{i}^{p} - {x}_{i}^{q} )}^{2}}$ |
| Manhattan | $d ( {X}^{p} , {X}^{q} ) = {\sum }_{i = 1}^{n} \vert {X}_{i}^{p} - {x}_{i}^{q} \vert$ |
| Maximum | d(X^{p}, X^{q}) = argmaxᵢ\|X₁^{p} − Xₙ^{q}\| |
| Value | |
| Normalized Euclidean | $d ( {X}^{p} , {X}^{q} ) = \sqrt{\frac{1}{n} {\sum }_{i = 1}^{n} {( \frac{{X}_{i}^{p} - {X}_{i}^{q}}{{max}_{i} - {min}_{i}} )}^{2}}$ |
| Normalized Manhattan | $d ( {X}^{p} , {X}^{q} ) = \frac{1}{n} {\sum }_{i = 1}^{n} \frac{\vert {X}_{i}^{p} - {X}_{i}^{q} \vert}{{max}_{i} - {min}_{i}}$ |
| Normalized Maximum Value | $d ( {X}^{p} , {X}^{q} ) = ar g {max}_{i} \frac{\vert {X}_{i}^{p} - {X}_{i}^{q} \vert}{{max}_{i} - {min}_{i}}$ |
| Dice Coefficient | $d ( {X}^{p} , {X}^{q} ) = 1 - \frac{2 {Σ}_{i - 1}^{n} {X}_{i}^{p} {X}_{i}^{q}}{{Σ}_{i - 1}^{n} {X}_{i}^{{p}^{2}} + {Σ}_{i - 1}^{n} {X}_{i}^{{q}^{2}}}$ |
| Cosine coefficient | $d ( {X}^{p} , {X}^{q} ) = 1 - \frac{{Σ}_{i - 1}^{n} {X}_{i}^{p} {X}_{i}^{q}}{\sqrt{{Σ}_{i - 1}^{n} {X}_{i}^{{p}^{2}} \cdot {Σ}_{i - 1}^{n} {X}_{i}^{{q}^{2}}}}$ |
| Jaccard coefficient | $d ( {X}^{p} , {X}^{q} ) = 1 - \frac{{Σ}_{i - 1}^{n} {X}_{i}^{p} {X}_{i}^{q}}{{Σ}_{i - 1}^{n} {X}_{i}^{{p}^{2}} + {Σ}_{i - 1}^{n} {X}_{i}^{{q}^{2}} - {Σ}_{i - 1}^{n} {X}_{i}^{p} {X}_{i}^{q}}$ |

Additional details and information regarding the distance based classification model of the respective classifier 222 can be learned from Yang et al., 1999, “DistAI: An Inter-pattern Distance-based Constructive Learning Algorithm,” Intelligent Data Analysis, 3(1), pg. 55.

In some embodiments, the plurality of heuristic instructions 224 include one or more heuristic instructions 224 for evaluating a candidate subject. For instance, in some embodiments, the plurality of heuristic instructions 224 for evaluating a candidate subject dictate how to parse a text object into one or more text strings, which form a plurality of information extracted from a respective communication 240. In some embodiments, one or more classifier 222 share one or more heuristic instructions 224.

In some embodiments, the classification system 200 includes a reference database 230 that stores one or more corpus of communications 232, hereinafter a “corpus” or a “corpus of communications.” In some embodiments, each corpus of communications 232 is associated with a unique candidate subject, which allows for the systems and methods of the present disclosure to combine information about the unique candidate subject at a single bin. In some embodiments, prior to applying a classifier 222 to a communication 240, a training set of data (e.g., a predetermined corpus of communication 232 of communications 240) is prepared to train the one or more classifiers 222. In some embodiments, the training set of data is a corpus of communications 232. In some embodiments, the corpus of communications 232 stores one or more communications 240 that each contain a specific tag 250, such as a first tag 250-1 associated with a particular class of assets (e.g., stable coin cryptocurrencies).

In some embodiments, other databases are communicatively linked (e.g., linked through the communication network 106 of FIG. 1) to the classification system 200. For instance, in some embodiments, one or more communications 240 stored on an external database stores (e.g., a cloud database, such as a database of clinical trials and/or intellectual property applications) is provided to the classification system 200 by way of the communications network 106.

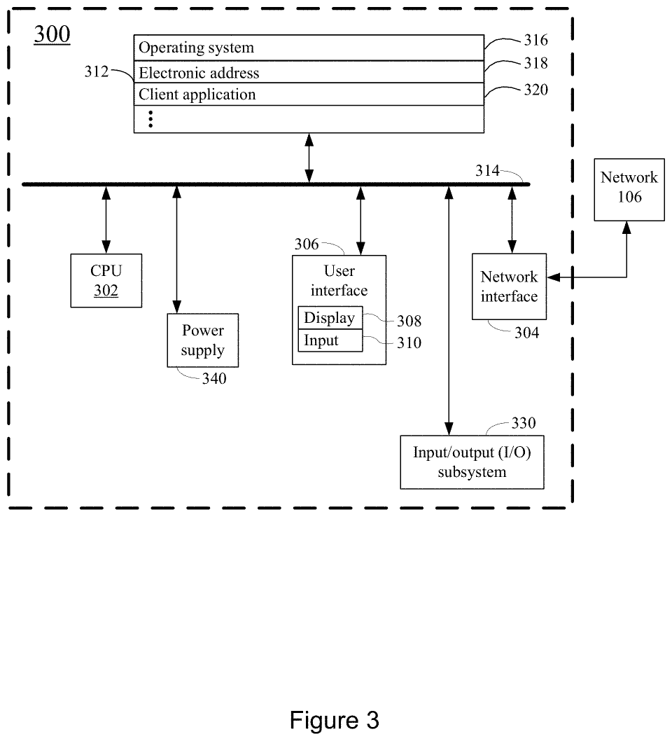

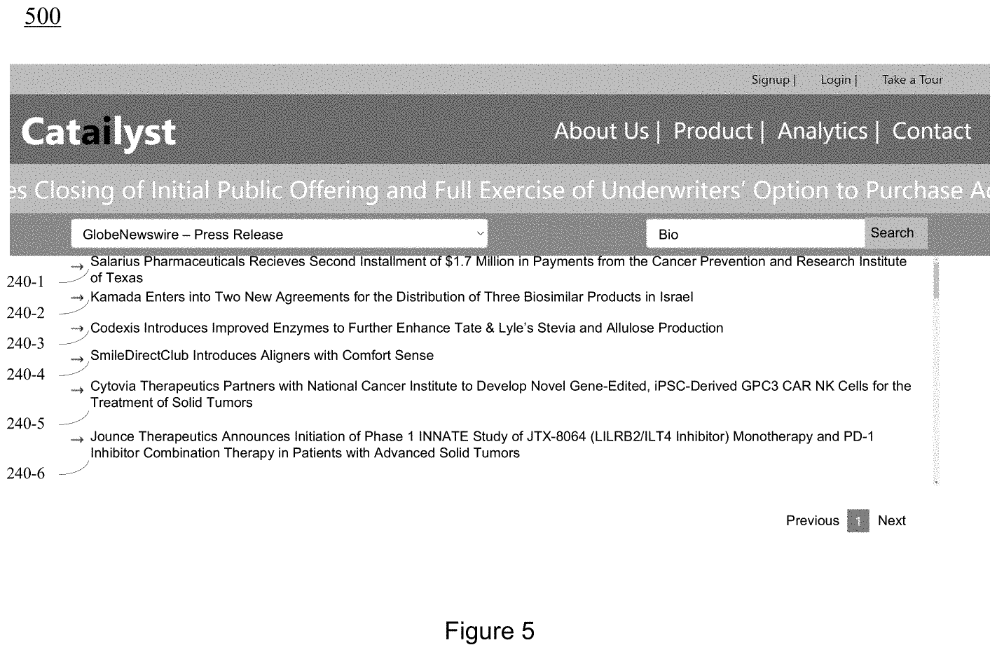

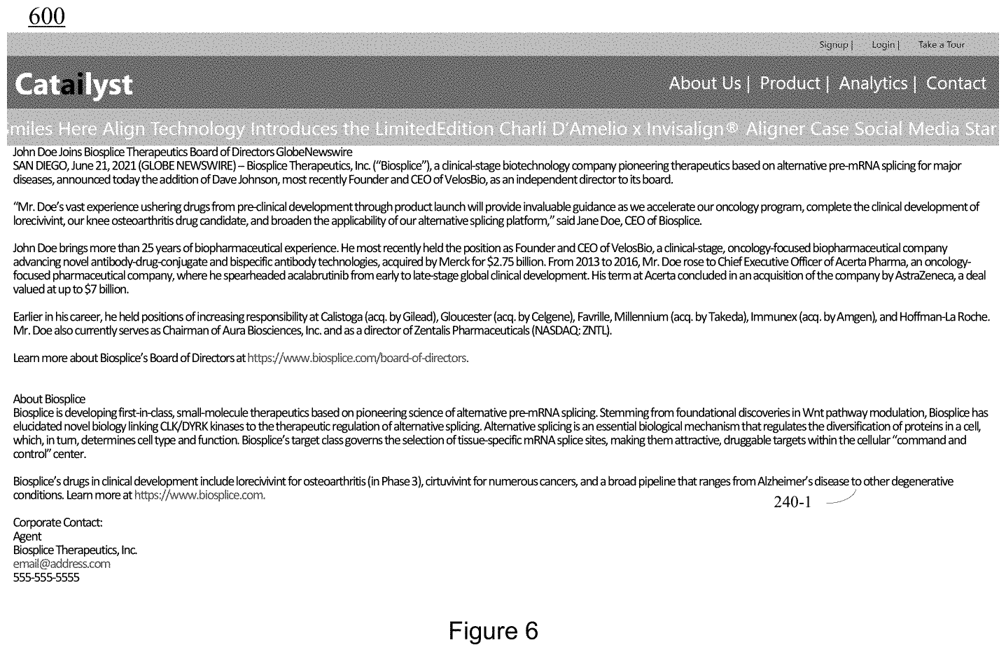

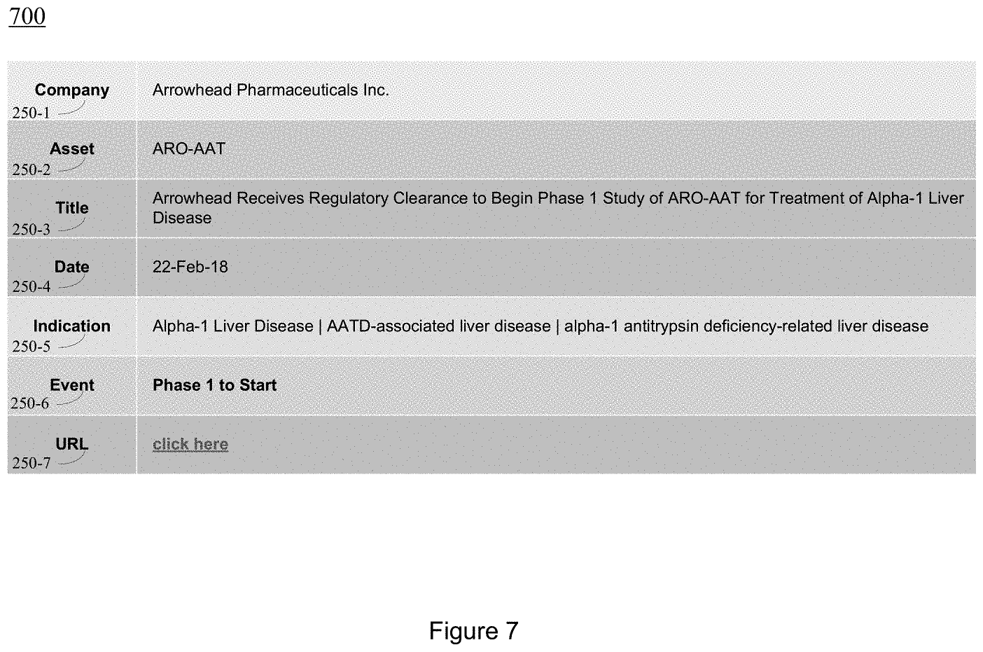

Furthermore, in some embodiments, the classification system 200 includes a reporting module 260 that facilitates providing an evaluation of a candidate subject a subject. For instance, in some embodiments, the reporting module 260 generates a user interface (e.g., user interface 306 of FIG. 3, user interface 500 of FIG. 5, user interface 600 of FIG. 6, user interface 700 of FIG. 7, etc.) for display at a client device 300. In some embodiments, the user interface generated by the reporting module 260 displays some or all of the corresponding plurality of information extracted by a respective classifier 222. In some embodiments, the user interface generated by the reporting module 260 displays some or all of the first communication 240-1. In some embodiments, the user interface generated by the reporting module 260 displays some or all of a corresponding corpus of communications 232.

In some embodiments, the reporting module 260 generates a report in response to a request for the report from a client device 400. In some embodiments, the request to generate the report is transmitted by the client device 400 on a recurring basis for a definite and/or indefinite period of time. In some embodiments, the recurring basis is a periodic basis. For instance, in some embodiments, the recurring basis is about 3 hours (e.g., 3.25 hours), about 6 hours, about 12 hours, about 24 hours, about 48 hours, about 5 days, about 7 days, about 30 days, about a month, quarterly, or a combination thereof. However, the present disclosure is not limited thereto. In some embodiments, the recurring basis is performed on a non-periodic basis, such as an irregularly timed basis.

In some embodiments, an account repository 270 retains a plurality of account constructs 272 (e.g., first account construct 272-1, second account construct 272-2, . . . , account construct S 272-S of FIG. 2). Each respective account construct 272 corresponds to an account held by a subject (e.g., user of a client device 300 of FIG. 3) with a service provider that is associated with the classification system 200 (e.g., provider of a client application 320 service of FIG. 3). In some embodiments, each respective account construct 272 includes a contact address of the user (e.g., electronic address 318 of client device 300 of FIG. 3). In some embodiments, each respective account construct 272 includes login information to access a service provided by the classification system, such as a service of the client application 320 of the client device 300.

In some embodiments, a user of the client device 300 defines a condition that causes the reporting module 260 to generate a report, which is then communicated to one or more client devices 300 associated with the user. In some embodiments, the condition defined by the user is retained in a corresponding account construct 272 associated with the user. In some embodiments, the condition is an indication for a condition, a clinical event (e.g., start of phase 1 trails, termination of clinical trials, etc.), an asset name, a regulatory event, a contractual obligation, or a company name.

In some embodiments, one or more of the above identified data stores and/or modules of the classification system 200 are stored in one or more of the previously described memory devices (e.g., memory 212), and correspond to a set of instructions for performing a function described above. The above-identified data, modules, or programs (e.g., sets of instructions) need not be implemented as separate software programs, procedures, or modules. Thus, various subsets of these modules may be combined or otherwise re-arranged in various implementations. In some embodiments, the memory 212 optionally stores a subset of the modules and data structures identified above. Furthermore, in some embodiments the memory 212 stores additional modules and data structures not described above.

Referring to FIG. 3, a description of an exemplary client device 300 that can be used with the presently disclosure is provided. In some embodiments, a client device 300 includes a smart phone (e.g., an iPhone, an Android device, etc.), a laptop computer, a tablet computer, a desktop computer, a wearable device (e.g., a smart watch, a heads-up display (HUD) device, etc.), a television (e.g., a smart television), or another form of electronic device such as a gaming console, a stand-alone device, and the like.

The client device 300 illustrated in FIG. 3 has one or more processing units (CPU's) 302, a network or other communications interface 304, a memory 312 (e.g., random access memory), a user interface 306, the user interface 306 including a display 308 and input 310 (e.g., keyboard, keypad, touch screen, etc.), an optional input/output (I/O) subsystem 330, and one or more communication busses 314 for interconnecting the aforementioned components.

In some embodiments, the input 310 is a touch-sensitive display, such as a touch-sensitive surface. In some embodiments, the user interface 306 includes one or more soft keyboard embodiments. In some embodiments, the soft keyboard embodiments include standard (QWERTY) and or non-standard configurations of symbols on the displayed icons. The input 310 and/or the user interface 306 is utilized by an end-user of the respective client device 300 (e.g., a respective subject) to input various commands (e.g., a push command) to the respective client device 300.

It should be appreciated that the client device 300 illustrated in FIG. 3 is only one example of a multifunction device that may be used for receiving one or more communications 240, generating one or more communications 240, transmitting one or more communications 240, analyzing a characteristic of one or more communications 240, or a combination thereof. Thus, the client device 300 optionally has more or fewer components than shown, optionally combines two or more components, or optionally has a different configuration or arrangement of the components. The various components shown in FIG. 3 are implemented in hardware, software, firmware, or a combination thereof, including one or more signal processing and/or application specific integrated circuits.

Memory 312 of the client device 300 illustrated in FIG. 3 optionally includes high-speed random access memory and optionally also includes non-volatile memory, such as one or more magnetic disk storage devices, flash memory devices, or other non-volatile solid-state memory devices.

There is an optional RF (radio frequency) circuitry of network interface 304 that may receive and send RF signals, also called electromagnetic signals. In some embodiments, the data constructs are received using the present RF circuitry from one or more devices such as client device 300 associated with a subject. In some embodiments, the network interface 304 converts electrical signals to from electromagnetic signals and communicates with communications networks (e.g., communication network 106 of FIG. 1) and other communications devices, client devices 300, and/or the classification system 200 via the electromagnetic signals. The network interface 304 optionally includes well-known circuitry for performing these functions, including but not limited to an antenna system, an RF transceiver, one or more amplifiers, a tuner, one or more oscillators, a digital signal processor, a CODEC chipset, a subscriber identity module (SIM) card, memory, and so forth. The network interface 304 optionally communicates with the communication network 106. In some embodiments, the network interface 304 does not include RF circuitry and, in fact, is connected to the communication network 106 through one or more hard wires (e.g., an optical cable, a coaxial cable, or the like).

In some embodiments, the memory 312 of the client device 300 stores:
an operating system 316 that includes procedures for handling various basic system services;
an electronic address 318 associated with the client device 300; and
a client application 320 for communicating a request for an evaluation of a candidate subject and/or visualizing the evaluation of the candidate subject through a graphical user interface.

As illustrated in FIG. 3, a client device 300 preferably includes an operating system 316 that includes procedures for handling various basic system services. The operating system 316 (e.g., iOS, ANDROID, DARWIN, RTXC, LINUX, UNIX, OS X, WINDOWS, or an embedded operating system such as VxWorks) includes various software components and or drivers for controlling and managing general system tasks (e.g., memory management, storage device control, power management, etc.) and facilitates communication between various hardware and software components.

An electronic address 318 is associated with each client device 300, which is utilized to at least uniquely identify the client device 300 from other devices and components of the integrated system 100. In some embodiments, the client device 300 includes a serial number, and optionally, a model number or manufacturer information that further identifies the client device 300. In some embodiments, the electronic address 318 associated with the client device 300 is used to provide a source of a communication 240 received from and/or provided to the client device 300.

A client application 320 is a group of instructions that, when executed by a processor (e.g., CPU(s) 302), generates content (e.g., a visualization of an evaluation of a candidate subject provided by the classification system 200) for presentation to the subject. In some embodiments, the client application 320 generates content in response to one or more inputs received from the subject through the user interface 306 of the client device 300. For instance, in some embodiments, the client application 320 includes a media presentation application for viewing the contents of a file or web application that includes the evaluation of the candidate subject.

In some embodiments, the client application 320 provides the same functionality as the classification model store 220, the reference database 230, the reporting module 260, the account repository 270, or a combination thereof of the classification system 200. In this way, in some embodiments, the client application 320 allows for an air-gapped classification and/or evaluation system without connections to an external network, such as the communication network 106.

In some embodiments, the client device 300 has any or all of the circuitry, hardware components, and software components found in the system depicted in FIG. 3. In the interest of brevity and clarity, only a few of the possible components of the client device 300 are shown to better emphasize the additional software modules that are installed on the client device 300.

Now that details of a system 100 for evaluating a lead development associated with a candidate subject based on one or more communications 240 have been disclosed, details regarding a flow chart of processes and features for implementing a method (e.g., method 400 of FIGS. 4A through 4F) for evaluating the candidate subject, in accordance with an embodiment of the present disclosure, are disclosed with reference to FIGS. 4A, 4B, 4C, 4D, 4E, and 4F.

Block 400. Referring to block 400 of FIG. 4A, a computer system (e.g., system 100 of FIG. 1, classification system 200 of FIG. 2, client device 300, etc.) for evaluating a candidate subject is provided. The computer system 100 includes one or more processors (e.g., CPU 202 of FIG. 2, CPU 302 of FIG. 3) and a memory (e.g., memory 212 of FIG. 2, memory 312 of FIG. 3). The memory 212 stores at least one program (e.g., classification model store 220 of FIG. 2, reference database 230 of FIG. 2, reporting module 260 of FIG. 2, account repository 270 of FIG. 2, client application 320 of FIG. 3, etc.). The at least the program includes one or more instructions for executing a method (e.g., method 400 of FIGS. 4A through 4F).

Block 402. Referring to block 402, the candidate subject is a topic of an evaluation that is based on information included in and/or derived from one or more communications 240. In this way, in some embodiments, the candidate subject is a subject matter, such as a broad topic including an industry (e.g., clinical and/or regulatory topic, financing topic, partnership topic, etc.). For instance, in some embodiments, the candidate subject is associated with a predetermined industry (e.g., a first candidate subject of a biotechnology industry, a second candidate subject of a pharmaceutical industry, a third candidate subject of a financial industry, a fourth candidate subject of a technology sector, etc.). In some embodiments, the candidate subject is selected from a group consisting of about 4 candidate subjects, about 6 candidate subjects, about 10 candidate subjects, about 15 candidate subjects, about 20 candidate subjects, about 25 candidate subjects, about 50 candidate subjects, about 75 candidate subjects, about 100 candidate subjects, about 150 candidate subjects, about 300 candidate subjects, about 500 candidate subjects, about 1,000 candidate subjects, or a combination thereof.

In this way, in some embodiments, a client device 300 associated with a user communicates a request for an evaluation of a candidate subject that is either defined by the user or selected from a listing of predetermined candidate subjects. However, the present disclosure is not limited thereto. For instance, in some embodiments, the candidate subject describes a topic that includes an entity, a tangible asset, an intangible asset, or a combination thereof. For instance, in some embodiments, the candidate subject of the entity includes a corporation (e.g., a candidate subject of a first limited liability corporation, a second candidate subject of a second limited liability partnership entity, etc.), a person (e.g., a public figure, an officer or an agent of a corporation, etc.), or both. As a non-limiting example, in some embodiments, the first candidate subject is a first corporation entity in a first industry, the second candidate subject is a second corporation entity in the first industry, a third candidate subject is a technology officer associated with the second entity, and a third candidate subject is the first industry. In some embodiments, the candidate subject includes a tangible asset, such as a consumer product (e.g., a candidate subject of a good, such as a toy; a commodity, etc.), a compound (e.g., a candidate subject of a class of pharmaceutical composition), a material (e.g., a candidate subject of a polymer), a tangible property (e.g., a candidate subject of a real estate property), or a combination thereof. In this way, the method 400 provides an evaluation of a specific, narrow candidate subject. For instance, in some embodiments, the intangible asset includes an intangible property (e.g., intellectual property such as a patent or copyright; a contract, etc.), a security (e.g., a stock, a bond, etc.), or both. In some embodiments, the tangible asset is a pharmaceutical product (e.g., a pharmaceutical composition). From this, the method 400 allows for an evaluation of a candidate subject that describes a broad topic, such as a respective characteristic of a specific entity; a narrow topic, such as a respective characteristic of a specific tangible assets; or both, such as an evaluation of the specific tangible assets that incorporates, or is based on, the specific entity. For instance, referring briefly to FIG. 5, a user interface 500 displays a plurality of communications 240 (e.g., first communication 240-1, second communication 240-2, . . . , sixth communication 240-6) of a corpus of communications 232 that are retrieved in response to a request for an evaluation of a candidate subject from a user of a client device, whereby the candidate subject is the term “Bio,” from a source “GlobeNewsWire,” in the form of “Press Release[s].” In some embodiments, the candidate subject is identified through a respective communication 240 that is received by the systems and methods of the present disclosure. By way of example, in some embodiments, a first communication 240-1 from a first source includes a plurality of text data that describes the first source starting production of a novel pharmaceutical composition. Accordingly, the method 400 identifies the novel pharmaceutical composition through the classifier 222 in order to form a candidate subject that is the novel pharmaceutical composition. In this way, the method 400 receives future communications 240 associated with the novel pharmaceutical composition and extracts information from these future communications 240 associated with the novel pharmaceutical composition.

In some embodiments, a user provides the candidate subject to the system 100 (e.g., the user communicates a request for an evaluation of the candidate subject through a client device 300). For instance, in some embodiments, the user provides a query to a classification system (e.g., classification system 200) for an evaluation of a first candidate subject. However, the present disclosure is not limited thereto. In some embodiments, this user provided candidate subject is then added to a listing of candidate subjects. In some embodiments, the system 100 determines the candidate subject for evaluation based on a determination formed from the query provided by the user. In some embodiments, the system 100 determines the candidate subject based upon an evaluation for a candidate subject in coordination with a reference database (e.g., reference database 230 of FIG. 2) and/or a trained classifier (e.g., trained classifier 222 of FIG. 2). For instance, in some embodiments, the method 400 compares a portion of a query with the reference database 230 and identifies a candidate subject based on this comparison. As a non-limiting example, consider a user providing a first query to the classification system 200 for an evaluation of “arrhythmia medication trends.” In response, the classification system 200 identifies a respective candidate subject of a calcium channel blocker pharmaceutical, a beta blocker pharmaceutical composition, a dietary treatment, a medical device treatment (e.g., pacemaker), or a combination thereof based on a comparison of the text data “arrhythmia medication trends” and one or more tags 250 associated with a plurality of communication 240 of the reference database 230, such as a corpus of communications 232 that includes one or more communications 240 associated with a topic of arrhythmia. As another example, in some embodiments, a second query includes a vague or ambiguous term within the text data of the second query, such as “What is an unmet need in the same field of this investment” that is actually associated with a first candidate subject. From this, in such embodiments, the method 400 identifies a second candidate subject for evaluation based on the vague query, such that the second candidate subject is identified solely through an identification of the first candidate subject.

Block 404. Referring to block 404, the method includes receiving a first communication (e.g., first communication 240-1 of FIG. 2) in a plurality of communications 240 in electronic form. As an electronic communication 240, in some embodiments, the first communication 240-1 is received in an unstructured form or a structured form, either of which includes a plurality of text data. In this way, in some embodiments, receiving the first communication 240-1 includes formatting the first communication 240-1 in accordance with a standardized format (e.g., modifying a format of the first communication from a first data format to a second data format). This formatting allows for seamless input into the classifier 222 regardless of a source of a communication 240. For instance, in some embodiments, a first communication 240-1 is received in electronic form in a portable document format (PDF), a second data construct is received in electronic form in a way format, and a third data construct is received in electronic form in a Hypertext Markup Language (HTML) electronic mail (Email) format. This seamless input is particularly useful for receiving a plurality of communications 240 in any variety of formats, irrespective of whether the communication includes unstructured text data or structured text data. Accordingly, in some embodiments, the system 100 formats each of the communications 240 into a predetermined format (e.g., standardized format, such as JSON) before applying the classifier 222. In some embodiments, formatting the communication 240 is in accordance with more than one standardized format (e.g., the communication 240 is formatted in a first standardized format, a second standardized format, or both). For instance, in some embodiments, the first communication 240-1 is formatted in a first format for use with a first classifier 222-1 and is further formatted in a second format for use with a second classifier 222-2. In some embodiments, this formatting of the communication 240 forms a transcript of one or more audio utterances of the communication 240. For instance, in some embodiments, the method 400 includes a data preparation module (e.g., a classifier 222 that includes a data preparation process), in which transcribing audio data of a communication 240 into a corresponding plurality of text data. In some embodiments, a speech-to-text classifier 222 assists with and/or provides the transcribing of the audio data of the communication 240. However, the present disclosure is not limited thereto. In some embodiments, a communication 240 includes a document (e.g., a paper document that is scanned to form an electronic document, or an electronic document such as a word document) that includes one or more text characters (e.g., text strings) which form the plurality of text data. For instance, a word document is a type of a communication 240, with the underlying data of the word document forming a plurality of text data. As another example, a recorded phone conversation is another type of a communication 240, with the transcribed text of the phone conversation and/or the audio data portion of the conversation forming a plurality of text data, and the transcribed text of the phone conversation forms the plurality of text data. In this way, in some embodiments, the plurality of text data is derived from a communication 240 (e.g., communication 240-1 of FIG. 7, communication 240-1 of FIG. 2, etc.). The communications 240 of the present disclosure include a variety of mechanisms for exchanging information (e.g., communicating) either through verbal forms (e.g., spoken communications 240), written (e.g., transcribed communications 240), and, in some embodiments, visual forms (e.g., graphical communications 240 such as charts and graphs). These mechanisms of communicating include text based documents (e.g., PDF's, word documents, spreadsheets, etc.,) and online platforms (e.g., communication 240 client application 320 of FIG. 3, social media feeds, text messages, online forums, blogs, review websites, etc.).

In some embodiments, a classifier 222 of the present disclosure processes a communication 240 to identify and/or amend an error (e.g., a clerical error such as a typo) within the communication 240. In such embodiments, if a communication 240 includes a type error (e.g., a clerical spelling error) or a semantic error, the type error or semantic error will propagate and force other errors in extracting information from the communication 240 or providing an evaluation of a candidate subject associated with the communication 240.

In some embodiments, the plurality of communications 240 includes at least 5 communications 240, at least 10 communications 240, at least 20 communications 240, at least 50 communications 240, at least 100 communications 240, at least 200 communications 240, at least 400 communications 240, at least 750 communications 240, at least 1,000 communications 240, at least 2,000 communications 240, at least 5,000 communications 240, at least 10,000 communications 240, at least 100,000 communications 240, at least 1,000,000 communications 240, or a combination thereof. As such, the systems and methods of the present disclosure allow for the receiving of a computationally substantial number of communications 240 that require a computer system (e.g., classification system 200 and/or client device 300) to be used because the communications cannot be evaluated mentally. Moreover, by using at least a defined plurality of communications 240 (e.g., at least 100 communications 240), the systems and methods of the present disclosure ensure a high level of accuracy and precise given the large sample size when forming a corpus of communications 232, which ensures an insightful evaluation of the candidate subject associated with the corpus of communications 232.

The communication 240 is an exchange of information from a source (e.g., client device 300, a remote server, etc.). Typically, the communication 240 provides the information in a human readable format, such as a language and/or a collection of figures. For instance, a respective communication 240 includes a release of information (e.g., a press release, a media release, etc.), a filing of information (e.g., a filing with and/or from an entity, such as a filing from a first entity with a second entity or a government filing), or a miscellaneous release of information, such as a raw data source (e.g., an un-curated database), a result from a clinical study, a market exchange information (e.g., a data packet received from an exchange platform, such as the Chicago Mercantile Exchange), and the like. For instance, referring briefly to FIG. 6, a first communication 240-1 includes a press release of information associated with a Mr. John Doe added to a board of directors at a company.

Each communication 240 in the plurality of communications 232-1 includes a respective plurality of text data. The plurality of text data conveys information (e.g, facts and/or opinions) of the communication 240. In some embodiments, the plurality of text data includes at least 50 characters, at least 100 characters, at least 500 characters, at least 1,000 characters, at least 2,000 characters, at least 5,000 characters, at least 7,500 characters, at least 10,000 characters, at least 15,000 characters, at least 25,000 characters, at least 50,000 characters, at least 100,000 characters, or a combination thereof. In this way, the systems and methods of the present disclosure 100 allow for the extraction of information from a substantially large collection of text data. As such, the systems and methods of the present disclosure require a computer system to be used because they cannot be mentally solved.

For instance, consider a first communication 240-1 that is a scholarly publication. As such, a corresponding plurality of text data of the first communication 240 includes a title of the scholarly publication, citation information of the publication (e.g., publication information of the scholar publication, an appendix of references of the scholarly publication, etc.), an abstract of the scholarly publication, a body of the scholarly publication, a figure of the scholarly publication, or a combination thereof, which conveys the information of the first communication 240-1. As another non-limiting example, consider a second communication 240-2 that is an official filing of a Form 10-Q filing with the Securities and Exchange Commission (SEC). As such, a corresponding plurality of text data of the second communication 240 includes a selection of one or more fields of the 10-Q (e.g., a first selection of a quarterly report field or a second selection of a transition report field; a respective selection of a filer type field; etc.), an entry of one or more fields of the 10-Q (e.g., a first entry of a file number field, a second entry of a jurisdiction of incorporation field, etc.), which conveys the information of the second communication 240-2. In some embodiments, the plurality of text data is written and/or authored by a human user.

In some embodiments, the first communication 240-1 is associated with the candidate subject. For instance, in some embodiments, the first communication 240-1 relates to a pharmaceutical composition belonging to a first class of compositions, such that a candidate subject includes the first class of compositions. In some embodiments, this relation is not directly communicated by the information of the first communication 240-1 (e.g., the relation is extracted by a classifier 222) or is extracted from the information of the first communication 240-1. As a non-limiting example, consider a first communication 240-1 that is a scholarly publication associated with a first pharmaceutical composition, that a first entity owns the first pharmaceutical composition, and that a candidate subject of an evaluation is the first entity. Accordingly, if the first communication 240-1 specifically describes the first entity with respect to the first pharmaceutical composition, the association between the first entity and the first communication 240-1 is directly communicated by the information of the first communication 240-1. On the other hand, if the first communication 240-1 only describes the first pharmaceutical composition and is otherwise silent with respect to the first entity, the association between the first entity and the first communication 240-1 is extracted from the information of the first communication 240-1. In some embodiments, this extracted associated is determined based on a predetermined association, such as a tag 250 of a respective communication 240 that describes the predetermined association of the reference database 230. As another non-limiting example, in some embodiments, this extracted association is based on a plurality of information of the first communication 240-1 and a second communication 240-2 (e.g., the second communication 240-2 describes the first entity owning the first pharmaceutical composition).

Block 406. Referring to block 406, in some embodiments, the method 400 conducts the receiving of the first communication 240-1 in response to a request to evaluate the candidate subject. For instance, in some embodiments, a client device 300 communicates a request to evaluate a specific candidate subject (e.g., a request to evaluate a first class of pharmaceutical compositions) to a classification system (e.g., classification system 200 of FIG. 2). In some embodiments, the request is in the form of an application programming interface (API) call. Accordingly, in some embodiments, response to receiving this request to evaluate the candidate subject, the method 400 receives the first communication 240-1 by polling for a publication of the first communication 240-1 from one or more public sources. In this way, by receiving the first communication 240-1 responsive to the request to evaluate the candidate subject, the method 400 provides the most recent and up-to-date information regarding the candidate subject, which ensures accuracy of the evaluation.

Block 408. Referring to block 408, in some embodiments, prior to receiving the first communication 240-1, the method 400 includes polling for the first communication 240-1 based on the association with the candidate subject. For instance, in some embodiments, the classification system 200 polls for a plurality of communications 240 from one or more remote devices (e.g., client device 300 of FIG. 3, a remote server, etc.). When a determination has been made that the first communication 240-1 exists, the method 400 receives the first communication 240-1. As a non-limiting example, consider a classification system 200 polling one or more remote devices for a first communication 240-1 associated with a first candidate subject that is a pharmaceutical composition. As such, the first candidate subject must be associated with at least the first communication 240-1 since the strict industry of pharmaceutical requires publishing a communication 240 when a regulatory event occurs, such as a publication of a Food and Drug Administration decision related to the pharmaceutical composition. In this way, the method 400 polls for the first communication 240-1, such that when the regulatory event occurs and the decision is published (e.g., the first communication 240-1 comes into existence), the first communication 240-1 is received by the classification system 200. However, the present disclosure is not limited thereto.

In some embodiments, the polling of the first communication 240-1 occurs by communicating with one or more remote databases, such as a first data base that includes candidate subject-specific aggregations of information, such as SEC corporate filings, medical databases, patent records, etc. In some embodiments, the polling of the first communication 240-1 occurs by communicating with an internal site that includes searchable databases for the internal communications 240 of one or more sites that are dynamically created, such as a knowledge base on a corporate site. In some embodiments, the polling of the first communication 240-1 occurs by communicating with one or more publication sources that includes searchable databases for current and archived communications 240. In some embodiments, the polling of the first communication 240-1 occurs by communicating with auction houses and/or shopping service providers, such as a classified listing. In some embodiments, the polling of the first communication 240-1 occurs by communicating with a portal that includes more than one of these other categories in searchable databases. In some embodiments, the polling of the first communication 240-1 occurs by communicating with one or more computation models, such as a database that includes an internal data component for determining one or more results including a mortgage computational module, dictionary look-ups computational module, and a translator between human languages computational model, or the like. Additional details and information regarding the receiving of a communication 240 can be found at Bergman, M., 2001, “White Paper: The Deep Web: Surfacing Hidden Value,” Journal of Electronic Publishing, 7(1), print; Dumbacher et al., 2018, “SABLE: Tools for Web Crawling, Web Scraping, and Text Classification,” Federal Committee on Statistical Methodology Research Conference, print; Yan, Y., 2016, “Text Analysis on SEC Filings (A Course Proposal),” print; Rosenfelder et al., 2017, Bayesian Modeling and Advanced Topics in Optimization (Seminar)—Preprocessing Text Data for Sentiment Analysis in R and Python,” print, each of which is hereby incorporated by reference in its entirety.

Block 410. Referring to block 410, in some embodiments, the request to evaluate the candidate subject is provided by a remote device, such as client device (e.g., client device 300 of FIG. 3). However, the present disclosure is not limited thereto. For instance, in some embodiments, the request to evaluate the candidate subject is generated locally at the classification system 200.

Block 412. Referring to block 412, in some embodiments, the request to evaluate the subject is provided (e.g., communicated through communications network 106 of FIG. 1) on a recurring basis for a definite and/or indefinite period of time. In some embodiments, the recurring basis is a periodic basis that occurs in repeated cycles. For instance, in some embodiments, the recurring basis is about 3 hours (e.g., 3.25 hours), about 6 hours, about 12 hours, about 24 hours, about 48 hours, about 5 days, about 7 days, about 30 days, about a month, quarterly, or a combination thereof. However, the present disclosure is not limited thereto. In some embodiments, the recurring basis is performed on a non-periodic basis, such as an irregularly timed basis.

Block 414. Referring to block 414, in some embodiments, the first communication 240-1 is received from a predetermined remote source. For instance, in some embodiments, the method 400 polls the predetermined remote source for one or more communications 240. In response to detecting the first communication 240-1, the system then receives the first communication 240-1 from the first source.

Block 416. Referring to block 416, the method 400 further includes extracting a corresponding plurality of information from the respective text data of the first communication 240-1. The extraction of the information from the first communication 240-1 is conducted by a trained classifier (e.g., classifier 222-1 of FIG. 2). In some embodiments, the trained classifier 224 in coordination with the reference database 230 further conducts the extraction. Referring briefly to FIGS. 6 and 7, user interfaces 600 and 700 depict different displays of the corresponding plurality of information extracted from the respective text data of the first communication 240-1. Specifically, referring to FIG. 6, a report is provided (e.g., by reporting module 260 of FIG. 2) that includes a title of the first communication 240-1, a summary of the first communication 240-1, a source of the first communication 240-1, and additional (e.g., other) information about the first communication 240-1. However, the present disclosure is not limited thereto. For instance, referring to FIG. 7, a report is provided that includes a name of an entity associated with the first communication 240-1 and a corresponding first tag 250-1, a name of an asset associated with the first communication 240-1 and a corresponding second tag 250-2, a title of the first communication 240-1 and a corresponding third tag 250-3, a publication date of the first communication 240-1 and a corresponding fourth tag 250-4, an indication associated with the first communication 240-1 and a corresponding fifth tag 250-5, an event associated with the first communication 240-1 and a corresponding sixth tag 250-6, and a source of the first communication 240-1 and a corresponding seventh tag 250-7.

More particularly, in some embodiments, the method 400 includes one or more instructions for training a classifier 222 (e.g., one or more partially trained or untrained classifiers 222) based on a feature data from a training dataset that includes one or more corpus of communications 232. In such embodiments, the feature data includes a characteristic of a candidate subject or the candidate subject.

In some embodiments, a probabilistic model is used in the methods and systems described herein, e.g., as a component model of an ensemble classifier 222. Probabilistic models employ random variables and probability distributions to a model for a phenomenon, e.g., the presence of a feature state, fraction, etc. Probabilistic models provide a probability distribution as a solution. Generally, probabilistic models can be classified as either graphical models (such as Bayesian networks, causal inference models, and Markov networks) or Stochastic models.

Probabilistic graphical models (PGMs) are probabilistic models for which a graph expresses a conditional dependence structure between random variables, encoding a distribution over a multi-dimensional space. One type of PGM is a Bayesian network, which is probabilistic graphical model that represents a set of variables and their conditional dependencies via a directed acyclic graph (DAG), according to Bayesian analysis. Briefly, given data x and parameter θ, Bayesian analysis uses a prior probability (a prior) p(θ) and a likelihood p(x|θ) to compute a posterior probability p(θ|x)∝p(x|θ)p(θ). Methods for learning Bayesian Networks are described, for example, in Castillo E, et al., “Learning Bayesian Networks,” Expert Systems and Probabilistic Network Models, Monographs in computer science, New York: Springer-Verlag, pp. 481-528, ISBN 978-0-387-94858-4, which is incorporated herein by reference, in its entirety, for all purposes. Another type of PGM is a Markov network, which is a set of random variables having a Markov property described by an undirected graph. Markov properties include pairwise Markov properties, in which any two non-adjacent variables are conditionally independent given all other variables, local Markov properties, in which a variable is conditionally independent of all other variables given its neighbors, and global Markov properties, in which any two subsets of variables are conditionally independent given a separating subset.

Stochastic probabilistic models model pseudo-randomly changing systems, assuming that future states depend only on a current state, not the events that occurred before the current state, otherwise known as the Markov property. Stochastic probabilistic models include Markov chains and Hidden Markov models (HMM). Markov chains are models describing a sequence of possible events in which the probability of each event depends only on the state attained in the previous event. For information on learning and application of Markov chains see, for example, Gagniuc, Paul A. (2017). Markov Chains: From Theory to Implementation and Experimentation. USA, NJ: John Wiley & Sons. pp. 1-235. ISBN 978-1-119-38755-8, which is incorporated herein by reference, in its entirety, for all purposes. Hidden Markov models (HMM) assume that a property X is dependent upon an unobservable (“hidden”) state Y that can be learned based on observation of the property. For review of Hidden Markov models see, for example, Rabiner and Juang, “An introduction to hidden Markov models,” IEEE ASSP Magazine, 3(1):4-16 (1986), which is incorporated herein by reference, in its entirety, for all purposes.

In some embodiments, a deep learning model, is used as a classifier 222 in the methods and systems described herein, e.g., as a component model of an ensemble classifier or circulating tumor fraction estimation model. Deep learning models use multiple layers to extract higher-level features from input data.

Neural networks. In some embodiments, the deep learning model of the classifier 222 is a neural network (e.g., a convolutional neural network and/or a residual neural network). Neural network algorithms, also known as artificial neural networks (ANNs), include convolutional and/or residual neural network algorithms (deep learning algorithms). Neural networks can be machine learning algorithms that may be trained to map an input data set to an output data set, where the neural network comprises an interconnected group of nodes organized into multiple layers of nodes. For example, the neural network architecture may include at least an input layer, one or more hidden layers, and an output layer. The neural network may include any total number of layers, and any number of hidden layers, where the hidden layers function as trainable feature extractors that allow mapping of a set of input data to an output value or set of output values. As used herein, a deep learning algorithm (DNN) can be a neural network that includes a plurality of hidden layers, e.g., two or more hidden layers. In some embodiments, each layer of the neural network includes a number of nodes (or “neurons”). A node can receive input that comes either directly from the input data or the output of nodes in previous layers, and perform a specific operation, e.g., a summation operation. In some embodiments, a connection from an input to a node is associated with a parameter (e.g., a weight and/or weighting factor). In some embodiments, the node may sum up the products of all pairs of inputs, xi, and their associated parameters. In some embodiments, the weighted sum is offset with a bias, b. In some embodiments, the output of a node or neuron is gated using a threshold or activation function, f, which may be a linear or non-linear function. The activation function may be, for example, a rectified linear unit (ReLU) activation function, a Leaky ReLU activation function, or other function such as a saturating hyperbolic tangent, identity, binary step, logistic, arcTan, softsign, parametric rectified linear unit, exponential linear unit, softPlus, bent identity, softExponential, Sinusoid, Sine, Gaussian, or sigmoid function, or any combination thereof.

The weighting factors, bias values, and threshold values, or other computational parameters of the neural network, may be “taught” or “learned” in a training phase using one or more sets of training data, such as a corpus of communications 232 associated with a particular candidate subject. For example, in some embodiments, the parameters is trained using the input data from a training data set (e.g., first corpus of communications 232-1 of FIG. 2) and a gradient descent or backward propagation method so that the output value(s) that the ANN computes are consistent with the examples included in the training data set. In some embodiments, the parameters are obtained from a back propagation neural network training process.

Any of a variety of neural networks may be suitable for use in extracting the corresponding plurality of information from the respective text data of the first communication 240-1 (e.g., block 416 of FIG. 4A), assigning a tag to each respective information in a subset of information of the corresponding plurality of information (e.g., block 436 of FIG. 4C), the applying the subset of tags to obtain an evaluation (e.g., block 454 of FIG. 4E), or a combination thereof. Examples can include, but are not limited to, feedforward neural networks, radial basis function networks, recurrent neural networks, residual neural networks, convolutional neural networks, residual convolutional neural networks, and the like, or any combination thereof. In some embodiments, the machine learning makes use of a pre-trained and/or transfer-learned ANN or deep learning architecture. Convolutional and/or residual neural networks can be used for in extracting the corresponding plurality of information from the respective text data of the first communication 240-1 (e.g., block 416 of FIG. 4A), assigning the tag to each respective information in the subset of information of the corresponding plurality of information (e.g., block 436 of FIG. 4C), the applying the subset of tags to obtain the evaluation (e.g., block 454 of FIG. 4E), or the combination thereof.

For instance, a deep neural network model includes an input layer, a plurality of individually parameterized (e.g., weighted) convolutional layers, and an output scorer. The parameters (e.g., weights) of each of the convolutional layers as well as the input layer contribute to the plurality of parameters (e.g., weights) associated with the deep neural network model. In some embodiments, at least 100 parameters, at least 1,000 parameters, at least 2,000 parameters or at least 5,000 parameters are associated with the deep neural network model. As such, deep neural network models require a computer to be used because they cannot be mentally solved. In other words, given an input to the model, the model output needs to be determined using a computer rather than mentally in such embodiments. See, for example, Krizhevsky et al., 2012, “Imagenet classification with deep convolutional neural networks,” in Advances in Neural Information Processing Systems 2, Pereira, Burges, Bottou, Weinberger, eds., pp. 1097-1105, Curran Associates, Inc.; Zeiler, 2012 “ADADELTA: an adaptive learning rate method,” CoRR, vol. abs/1212.5701; and Rumelhart et al., 1988, “Neurocomputing: Foundations of research,” ch. Learning Representations by Back-propagating Errors, pp. 696-699, Cambridge, Mass., USA: MIT Press, each of which is hereby incorporated by reference.

Neural network algorithms, including convolutional neural network algorithms, suitable for use as models are disclosed in, for example, Vincent et al., 2010, “Stacked denoising autoencoders: Learning useful representations in a deep network with a local denoising criterion,” J Mach Learn Res 11, pp. 3371-3408; Larochelle et al., 2009, “Exploring strategies for training deep neural networks,” J Mach Learn Res 10, pp. 1-40; and Hassoun, 1995, Fundamentals of Artificial Neural Networks, Massachusetts Institute of Technology, each of which is hereby incorporated by reference. Additional example neural networks suitable for use as models are disclosed in Duda et al., 2001, Pattern Classification, Second Edition, John Wiley & Sons, Inc., New York; and Hastie et al., 2001, The Elements of Statistical Learning, Springer-Verlag, New York, each of which is hereby incorporated by reference in its entirety. Additional example neural networks suitable for use as models are also described in Draghici, 2003, Data Analysis Tools for DNA Microarrays, Chapman & Hall/CRC; and Mount, 2001, Bioinformatics: sequence and genome analysis, Cold Spring Harbor Laboratory Press, Cold Spring Harbor, N.Y., each of which is hereby incorporated by reference in its entirety.

In some embodiments, a mixture model, also referred to herein as an admixture model, is used as a classifier 222 in the methods and systems described herein, e.g., as a component model of a classifier 222. Mixture models are probabilistic model for representing the presence of subpopulations within an overall population, without requiring that an observed data set should identify the sub-population to which an individual observation belongs. Given a sampling of parameter data from a mixture of distributions, e.g., term occurrence, parts of speech, and financial model distributions of the parameters over each distribution separately, several techniques can be used to determine the parameters of the particular mixture of distributions. These techniques include maximum likelihood estimation (e.g., expectation maximization), application of Bayes' theorem on posterior sampling of the mixture of distributions (e.g., via a Markov chain Monte Carlo algorithm such as Gibbs sampling), moment matching, and several graphical methodologies. For a review of the use of mixture models see, for example, Titterington, D et al., “Statistical Analysis of Finite Mixture Distributions,” Wiley ISBN 978-0-471-90763-3 (1985), which is incorporated herein by reference, in its entirety, for all purposes.

Logistic regression algorithms suitable for use as classifiers 222 are disclosed, for example, in Agresti, An Introduction to Categorical Data Analysis, 1996, Chapter 5, pp. 103-144, John Wiley & Son, New York, which is hereby incorporated by reference.

Neural network algorithms, including convolutional neural network algorithms, suitable for use as classifiers 222 are disclosed in, for example, Vincent et al., 2010, “Stacked denoising autoencoders: Learning useful representations in a deep network with a local denoising criterion,” J Mach Learn Res 11, pp. 3371-3408; Larochelle et al., 2009, “Exploring strategies for training deep neural networks,” J Mach Learn Res 10, pp. 1-40; and Hassoun, 1995, Fundamentals of Artificial Neural Networks, Massachusetts Institute of Technology, each of which is hereby incorporated by reference. A neural network has a layered structure that includes a layer of input units (and the bias) connected by a layer of weights to a layer of output units. For regression, the layer of output units typically includes just one output unit. However, neural networks can handle multiple quantitative responses in a seamless fashion. In multilayer neural networks, there are input units (input layer), hidden units (hidden layer), and output units (output layer). There is, furthermore, a single bias unit that is connected to each unit other than the input units. Additional example neural networks suitable for use as classifiers 222 are disclosed in Duda et al., 2001, Pattern Classification, Second Edition, John Wiley & Sons, Inc., New York; and Hastie et al., 2001, The Elements of Statistical Learning, Springer-Verlag, New York, each of which is hereby incorporated by reference in its entirety. Additional example neural networks suitable for use as classifiers are also described in Draghici, 2003, Data Analysis Tools for DNA Microarrays, Chapman & Hall/CRC; and Mount, 2001, Bioinformatics: sequence and genome analysis, Cold Spring Harbor Laboratory Press, Cold Spring Harbor, N.Y., each of which is hereby incorporated by reference in its entirety.

SVM algorithms suitable for use as classifiers 222 are described in, for example, Cristianini and Shawe-Taylor, 2000, “An Introduction to Support Vector Machines,” Cambridge University Press, Cambridge; Boser et al., 1992, “A training algorithm for optimal margin classifiers,” in Proceedings of the 5^{th} Annual ACM Workshop on Computational Learning Theory, ACM Press, Pittsburgh, Pa., pp. 142-152; Vapnik, 1998, Statistical Learning Theory, Wiley, New York; Mount, 2001, Bioinformatics: sequence and genome analysis, Cold Spring Harbor Laboratory Press, Cold Spring Harbor, N.Y.; Duda, Pattern Classification, Second Edition, 2001, John Wiley & Sons, Inc., pp. 259, 262-265; and Hastie, 2001, The Elements of Statistical Learning, Springer, New York; and Furey et al., 2000, Bioinformatics 16, 906-914, each of which is hereby incorporated by reference in its entirety. When used for classification of textual data in a respective communication 240, SVMs separate a given set of binary labeled data training set (e.g., a first and second term condition of each respective term in a plurality of terms in a corpus of communications 232) with a hyperplane that is maximally distant from the labeled data. For cases in which no linear separation is possible, SVMs can work in combination with the technique of kernels, which automatically realize a non-linear mapping to a feature space. The hyperplane found by the SVM in feature space corresponds to a non-linear decision boundary in the input space.

Naïve Bayes classifiers suitable for use as classifiers 222 are disclosed, for example, in Ng et al., 2002, “On discriminative vs. generative classifiers: A comparison of logistic regression and naive Bayes,” Advances in Neural Information Processing Systems, 14, which is hereby incorporated by reference.

Decision trees algorithms suitable for use as classifiers 222 are described in, for example, Duda, 2001, Pattern Classification, John Wiley & Sons, Inc., New York, pp. 395-396, which is hereby incorporated by reference. Tree-based methods partition the feature space into a set of rectangles, and then fit a model (like a constant) in each one. In some embodiments, the decision tree is random forest regression. One specific algorithm that can be used as a classifier 222 is a classification and regression tree (CART). Other examples of specific decision tree algorithms that can be used as classifiers 222 include, but are not limited to, ID3, C4.5, MART, and Random Forests. CART, ID3, and C4.5 are described in Duda, 2001, Pattern Classification, John Wiley & Sons, Inc., New York. pp. 396-408 and pp. 411-412, which is hereby incorporated by reference. CART, MART, and C4.5 are described in Hastie et al., 2001, The Elements of Statistical Learning, Springer-Verlag, New York, Chapter 9, which is hereby incorporated by reference in its entirety. Random Forests are described in Breiman, 1999, “Random Forests—Random Features,” Technical Report 567, Statistics Department, U.C. Berkeley, September 1999, which is hereby incorporated by reference in its entirety.

Clustering algorithms suitable for use as classifiers 222 are described, for example, at pages 211-256 of Duda and Hart, Pattern Classification and Scene Analysis, 1973, John Wiley & Sons, Inc., New York, (hereinafter “Duda 1973”) which is hereby incorporated by reference in its entirety. As set forth in Section 6.7 of Duda 1973, the clustering problem is described as one of finding natural groupings in a dataset. To identify natural groupings, two issues are addressed. First, a way to measure similarity (or dissimilarity) between two samples is determined. This metric (similarity measure) is used to ensure that the samples in one cluster are more like one another than they are to samples in other clusters. Second, a mechanism for partitioning the data into clusters using the similarity measure is determined. Similarity measures are discussed in Section 6.7 of Duda 1973, where it is stated that one way to begin a clustering investigation is to define a distance function and to compute the matrix of distances between all pairs of samples in a dataset. If distance is a good measure of similarity, then the distance between samples in the same cluster will be significantly less than the distance between samples in different clusters. However, as stated on page 215 of Duda 1973, clustering does not require the use of a distance metric. For example, a nonmetric similarity function s(x, x′) can be used to compare two vectors x and x′. Conventionally, s(x, x′) is a symmetric function whose value is large when x and x′ are somehow “similar.” An example of a nonmetric similarity function s(x, x′) is provided on page 216 of Duda 1973.

Once a method for measuring “similarity” or “dissimilarity” between points in a dataset has been selected, clustering makes use of a criterion function that measures the clustering quality of any partition of the data. Partitions of the dataset that extremize the criterion function are used to cluster the data. See page 217 of Duda 1973. Criterion functions are discussed in Section 6.8 of Duda 1973. More recently, Duda et al., Pattern Classification, 2^{nd} edition, John Wiley & Sons, Inc. New York, has been published. Pages 537-563 describe clustering in detail. More information on clustering techniques suitable for use as classifiers are disclosed in Kaufman and Rousseeuw, 1990, Finding Groups in Data: An Introduction to Cluster Analysis, Wiley, New York, N.Y.; Everitt, 1993, Cluster analysis (3d ed.), Wiley, New York, N.Y.; and Backer, 1995, Computer-Assisted Reasoning in Cluster Analysis, Prentice Hall, Upper Saddle River, N.J. Particular exemplary clustering techniques that can be used as classifiers include, but are not limited to, hierarchical clustering (agglomerative clustering using nearest-neighbor algorithm, farthest-neighbor algorithm, the average linkage algorithm, the centroid algorithm, or the sum-of-squares algorithm), k-means clustering, fuzzy k-means clustering algorithm, and Jarvis-Patrick clustering.

In some embodiments, a classifier 222 is a nearest neighbor algorithm. For nearest neighbors, given a query point x₀ (a test subject), the k training points x₍ᵣ₎, r, . . . , k (here the training subjects) closest in distance to x₀ are identified and then the point x₀ is classified using the k nearest neighbors. Here, the distance to these neighbors is a function of the abundance values of the discriminating gene set. In some embodiments, Euclidean distance in feature space is used to determine distance as d₍ᵢ₎=|x₍ᵢ₎−x_{(O)}∥. Typically, when the nearest neighbor algorithm is used, the abundance data used to compute the linear discriminant is standardized to have mean zero and variance 1. The nearest neighbor rule can be refined to address issues of unequal class priors, differential misclassification costs, and feature selection. Many of these refinements involve some form of weighted voting for the neighbors. For more information on nearest neighbor analysis, see Duda, Pattern Classification, Second Edition, 2001, John Wiley & Sons, Inc; and Hastie, 2001, The Elements of Statistical Learning, Springer, New York, each of which is hereby incorporated by reference.

Block 418. Referring to block 418 of FIG. 4B, furthermore, in some embodiments, the first communication 240-1 is received from the first source (e.g., client device 300, a remote server, etc.). In this way, the first source is different from the classification system 200. Accordingly, prior to the extracting of the information (e.g., block 416 of FIG. 4A), the method 400 include instructions for validating the first source. By validating the first source, the method 400 ensures that the first communication 240-1 is received from a trust source that is known to provide trustworthy information.

In some embodiments, the first source is a remote database. In some embodiments, the first source is a remote database that includes one or more communications 240 associated with clinical trials, such as clinicaltrials.gov. In some embodiments, the first source is associated with a regulatory entity and/or database, such as FDA.gov. In some embodiments, the first source is a publisher, such as Pubmed or Harvard University Press. In some embodiments, the first source is a conference, such as an abstract from one or more presentations at an industry conference. In some embodiments, the first source is a transcript of an audio conversation including one or more human subjects, such as an invention disclosure meeting. In this way, the systems and methods of the present disclosure allow for receiving the plurality of communications 240 from the first source that acts as a curating for the plurality of communications. In some embodiments, this first source is further associated with one or more candidate subjects (e.g., the first source curates one or more communications that are associated with a subset of candidate subjects, such as any engineering related candidate subjects).

Block 420. Referring to block 420, in some embodiments, the validating the first source includes determining a type of source associated with the first source. As such, in accordance with the type of source of the first communication 240-1, the method 400 provides either a validation of the first communication 240-1 as including reliable information, or invalidation of the first communication 240-1 as including unreliable information. As a non-limiting example, in some embodiments, the type of source includes a primary source that gives direct evidence about a respective subject matter (e.g., candidate subject). In some embodiments, the type of source includes a secondary source that describes the respective subject matter from the primary source.

Block 422. Referring to block 422, in some embodiments, validating the first source includes receiving a validation of the first source from a human subject (e.g., a user associated with a client device 300 and/or the classification system 200). In some embodiments, the human subject is unassociated with the first source. In this way, the human subject does not impart an inherent bias when validating the first source by way of association with the first source. Furthermore, in some embodiments, the human subject is associated with the classification system 200, which allows for an impartial, unbiased validation of the first source.

Block 424. Referring to block 424, in some embodiments, the validating of the first source include assigning a weight of credibility to the first communication 240-1. For instance, consider a first communication 240-1 from a first source that includes a first information describing a profit of a first entity with two significant figures (e.g., $1.7 million of FIG. 6), whereas a second communication 240-2 from a second source includes the first information but describes the profit of the first entity with one significant figure (e.g., $2 million). In this way, the method 400 assigns a first weight to the first source and a second weight to the second source in which the first weight is greater than the second weight since the first source had a higher precision in reporting the profit, and, therefore, improved validating processes. Additional details and information regarding assigning a weight can be found at Žižović et al., 2019, “New Model for Determining Criteria Weights: Level Based Weight Assessment (LBWA) Model,” Decision Making: Applications in Management and Engineering, 2(2), pg. 126, which is hereby incorporated by reference in its entirety.

Block 426. Referring to block 426, in some embodiments, the type of source includes a press media (e.g., a publication from a multimedia news corporation), a news media (e.g., a blog post), a filing with an entity (e.g., a trademark application filing with the United States Patent and Trademark Office (USPTO); a 10-Q filing with the SEC, etc.), a release from the entity (e.g., a publication from a website associated with an entity), or a combination thereof. As described supra, in some embodiments, the entity includes a government entity (e.g., a patent filing with the USPTO, a 10-K filing with the SEC, etc.). In some embodiments, the entity includes a publication entity (e.g., a scientific publication with a scientific journal). In some embodiments, the entity includes a conference hosted by an entity (e.g., a scientific publication with a scientific journal). Accordingly, in some embodiments, if a source of the communication is determined to be of a predetermined plurality of sources, the classifier 222 considers a credibility of the source of the communication 240 when extracting information of the communication 240. For instance, in accordance with a determination that the source of the first communication is a government entity, the first communication 240-1 is validated. In this way, one or more communications 240 that is received from a trusted source is validated based on the trusted source alone, as opposed to a validation through the information of the first communication 240-1. However, the present disclosure is not limited thereto.

Block 428. Referring to block 428, in some embodiments, the corresponding plurality of information of the extracting contains a first portion of the text data. In some embodiments, the first portion of the text data is less than all of the text data. In this way, the extracting of the information excludes a second portion of the text data that is not pertinent to obtaining an evaluation of the candidate subject. In some embodiments, the first portion of the text data includes one or more predetermined portions of the first communication 240-1. For instance, referring briefly to FIGS. 6 and 7, a user interface that displays the extracting of the information shows that a portion of the first communication 240-1 was excluded, in order to reduce a cognitive burden on a user that requests an evaluation of a candidate subject associated with the first communication 240-1. In some embodiments, the first portion of the text data includes the most important information of the first communication 240-1, such as any necessary information required to convey the subject matter of the first communication 240-1. However, the present disclosure is not limited thereto.

Block 430. Referring to block 430, in some embodiments, the classifier 222 conducts the extraction of the plurality of information in accordance with a corresponding plurality of heuristic instructions (e.g., heuristic instructions 224 of FIG. 2) that is associated with the classifier 222 and/or the extracting conducted by the classifier 222. Accordingly, the corresponding plurality of heuristic instructions 222 describe how the classifier 222 conducts the extraction, such as on a parts of speech basis, on a statistical module basis, and the like (e.g., classifiers 222 of block 416 of FIG. 4A). For instance, in some embodiments, a first plurality of heuristic instructions 224-1 describes how a first classifier 222-1 searches a communication 240 for one or more predetermined words and then propagates the search to local regions of the communication (e.g., a range of 100 characters from where the word was identified, a paragraph containing the word, the previous and following paragraphs of the paragraph containing the word, etc.). In some embodiments, a second plurality of heuristic instructions 22402 describes how a second classifier 22202 identifies an abstract (e.g., by an evaluation of word count, by location within the communication 240, etc.) of the communication 240 and then extracts information from the abstract.

Block 432. Referring to block 432 of FIG. 4C, in some embodiments, the corresponding plurality of heuristic instructions 224 includes a first subset of heuristic instructions 224 that extracts the first plurality of text data of the first communication 240-1 into a first subset of information that contains the first portion of the corresponding plurality of information. For instance, in some embodiments, the first portion of the text data includes a title of the first communication 240-1, one or more headings (i.e., headers) of the first communication 240-1, one or more sub-headings (i.e., sub-headers) of the first communication 240-1, an abstract of the first communication 240-1, a predetermined number of characters of the first communication 240-1, a predetermined number of words of the first communication 240-1, or a combination thereof. In some embodiments, the predetermined umber of characters of the first communication 240-1 is the first 5 characters, the first 10 characters, the first 17 characters, the first 20 characters, the first 25 characters, the first 27 characters, the first 30 characters, the first 35 characters, the first 40 characters, the first 42 characters, the first 50 characters, the first 54 characters, the first 60 characters, the first 70 characters, or a combination thereof (e.g., first 52 characters). In some embodiments, the predetermined umber of characters of the first communication 240-1 is the final 5 characters, the final 10 characters, the final 17 characters, the final 20 characters, the final 25 characters, the final 27 characters, the final 30 characters, the final 35 characters, the final 40 characters, the final 42 characters, the final 50 characters, the final 54 characters, the final 60 characters, the final 70 characters, or a combination thereof (e.g., 52 characters). Accordingly, the corresponding plurality of heuristic instructions 224 provide instructions for the classifier 224 on extracting the first portion of the text data including extracting the title of the first communication 240-1, the one or more headings (i.e., headers) of the first communication 240-1, the one or more sub-headings (i.e., sub-headers) of the first communication 240-1, the abstract of the first communication 240-1, the predetermined number of characters of the first communication 240-1, the predetermined number of words of the first communication 240-1, or a combination thereof. In some embodiments, the corresponding plurality of heuristic instructions 224 includes a second subset of heuristic instructions 224 that extracts a second portion of the text data of the first communication 240-1 into a second subset of information that contains a second portion of the corresponding plurality of information. In some embodiments, the second portion of the corresponding plurality of information includes a some or all of a body of the first communication 240-1.

Block 434. Referring to block 434, in some embodiments, the first subset of information and the second subset of information are disjoint subsets of the corresponding plurality of information, such that each respective subject of information includes unique information. In this way, a computational burden is reduced when storing and evaluating each respective subject of information that within a corpus of communications 232. However, the present disclosure is not limited thereto.

Block 436. Referring to block 436 of FIG. 4C, the method 400 further includes assigning a tag (e.g., first tag 250-1 of FIG. 2) to each respective information in a subset of information of the corresponding plurality of information. Each tag 250 is associated with a descriptor or aspect of a candidate subject, such that when a communication 240 is assigned a respective tag 250 (e.g., fourth tag 250-4 of FIG. 7), the communication 240 is considered to be associated with that description of aspect associated with the respective tag 250. By assigning the tag 250 to each respective information, the method 400 collectively assigns a first plurality of tags 250 in the set of tags 250 to the corresponding plurality of information. In this way, the first plurality of tags 250 assigned to the first communication 240 provide an overview of the information extracted from the first communication 240-1 by the classifier 222. From this, in some embodiments, a credibility of two or more communications 240 is considered based on a comparison of a respective first plurality of tags 250 assigned to each corresponding communication 240.

Block 438. Referring to block 438, in some embodiments, prior to receiving the first communication 240-1, the method 400 includes training the classifier 222 to evaluate the communication 240 based on the corpus of communications 232 (e.g., forming a trained classifier 222 based on the corpus of communications 232). In this way, the classifier 222 becomes trained to produce an evaluation for a particular candidate subject and/or tag 250. In some embodiments, the classifier 222 is trained with human supervision. In some embodiments, the classifier 222 is tried without human interference.

Block 440. Referring to block 440, in some embodiments, the corpus of communications 232 is associated with the candidate subject. For instance, in some embodiments, each respective corpus of communications 232 is associated with a type of candidate subject, such as a particular industry or a class of a product (e.g., a class of a pharmaceutical composition). In this way, the corpus of communications 232 is enabled to receive one or more communications 240, extract information from the one or more communications 240 associated with a candidate subject by way of the classifier 222, and store this extracted information in the corpus of communications 232 associated with the candidate subject.

Block 442. Referring to block 442 of FIG. 4D, in some embodiments, the corpus of communication 232 is uniquely associated with the candidate subject. For instance, in some embodiments, a first corpus of communications 232-1 is associated with a first candidate subject (e.g., associated with a first candidate subject of a first pharmaceutical composition) and a second corpus of communications 232-2 is associated with a second candidate subject (e.g., associated with a second candidate subject of a second pharmaceutical composition). In this way, each respective corpus of communications 232 becomes a subject matter expect for information of any communication associated with a corresponding candidate subject of a respective corpus of communications 232.

Block 444. Referring to block 444, in some embodiments, method 400 includes adding the first communication 240-1 to the corpus of communications 232. In this way, the reference database 230 dynamically updates to incorporate the first communication 240-1 when the first communication 240-1 is published, such that an evaluation of a second communication 240-2 is more robust based on the storing of the first communication 240-1 in the reference database 230. In some embodiments, the corpus of communications 232 includes the corresponding plurality of information of the first communication 240-1. In some embodiments, the corpus of communications 232 includes the first plurality of tags 250 of the first communication 240-1. From this, the corpus of communications 232 retains information extracted by the classifier 222, allowing the extracted information to be used in obtaining an evaluation of a candidate subject.

Block 446. Referring to block 446, in some embodiments, the corpus of communications 232 includes the corresponding plurality of information of the first communication, the first plurality of tags 250 of the first communication 250, or both. In this way, the corpus of communications 232 stores the tags 250 (e.g., first column of FIG. 7) and/or the information (e.g., second column of FIG. 7) that is extracted and/or assigned to the first communication 240-1. In this way, the method 400 is enabled to aggregated and compile the extracted information associated with a candidate subject to provide a robust data set to conduct evaluations thereon.

Block 448. Referring to block 448, in some embodiments, the text data of the first communication 240-1 includes unstructured text data, which includes information that either does not have a pre-defined data structure and/or is not organized in a predefined manner. By way of example, in some embodiments, information in an SEC filing is substantially unstructured text data. In some embodiments, the receiving of the first communication 240-1 further includes parsing the unstructured text data for use with the classifier 222. In some embodiments, the parsing of the receiving the first communication 240-1 is conducted by the classifier 222 (e.g., the trained classifier 222 includes one or more natural language processing classification modules).

In some embodiments, the set of tags 250 includes at least 12 tags 250, at least 15 tags 250, at least 20 tags 250, at least 25 tags 250, at least 30 tags 250, at least 40 tags 250, at least 50 tags 250, at least 60 tags 250, at least 70 tags 250, at least 80 tags 250, at least 90 tags 250, at least 100 tags 250, at least 150 tags 250, at least 200 tags 250, at least 250 tags 250, at least 300 tags 250, at least 400 tags 250, at least 500 tags 250, at least 600 tags 250, at least 700 tags 250, at least 800 tags 250, at least 900 tags 250, at least 1,000 tags 250, or a combination thereof.

In some embodiments, the first plurality of tags 250 in the set of tags 250 includes at least 2 tags 250, at least 3 tags 250, at least 4 tags 250, at least 5 tags 250, at least 7 tags 250, at least 10 tags 250, at least 15 tags 250, at least 20 tags 250, at least 25 tags 250, at least 30 tags 250, at least 40 tags 250, at least 50 tags 250, or a combination thereof. In this way, the set of tags 250 forms a pool of tags 250, whereby a subset of tags 250 in the set of tags 250 that is the first plurality of tags is applicable to a respective communication 250 in the plurality of communications 240.

In some embodiments, the set of tags 250 includes a subset of tier tags 250. For instance, in some embodiments, the subset of tier tags 250 includes one or more first tier tag 250, one or more second tier tags 250, and, optionally, one or more third tier tags 250. As such, in some embodiments, the first subset of information is assigned a first tier tag 250 in the subset of tier tags 250. In some embodiments, the second subset of information is associated with a second tier tag 250 in the subset of tier tags 250. In this way, the first subset of information is considered pertinent in providing an evaluation of the candidate subject, and the second subset of information is considered pertinent in providing an evaluation of the candidate subject, but less pertinent than the first subset of information and/or based on the first subset of information. From this, in some embodiments, the second tier tag 250 is lower than the first tier tag 250 in the plurality of tier tags 250 and/or based on the first tier tag 250 (e.g., based on the first subset of information). As a lower tier, the second tier tags 250 provide more granular classification of information in comparison to the first tier tags 250. More instance, in some embodiments, the first tier tags are associated with a class of pharmaceutical compositions and the second tier tags are associated with particular pharmaceutical compositions in the class of pharmaceutical compositions. However, the present disclosure is not limited thereto.

In some embodiments, the first trier tags 250 includes at least 5 tags 250, at least 7 tags 250, at least 10 tags 250, at least 15 tags 250, at least 25 tags 250, at least 50 tags, at least 100 tags, at least 1,000 tags, or a combination thereof. In some embodiments, the second trier tags 250 includes at least 2 tags 250, at least 3 tags 250, at least 4 tags 250, at least 5 tags 250, at least 7 tags 250, at least 10 tags 250, at least 15 tags 250, at least 20 tags 250, at least 50 tags 250, at least 100 tags 250, or a combination thereof. In some embodiments, the third trier tags 250 includes at least 2 tags 250, at least 3 tags 250, at least 4 tags 250, at least 5 tags 250, at least 7 tags 250, at least 10 tags 250, at least 15 tags 250, at least 20 tags 250, or a combination thereof.

Block 450. Referring to block 450, in some embodiments, the set of tags 250 includes a subset of category tags 250. In some embodiments, the assigning includes a respective category tag 250 in the subset of category tags to the corresponding of information. In this way, the method 400 determines a broad category of the first communication 240-1 and then assigns a respective category tag 250 in the subject of category tags 250. From this, in some embodiments, the classifier 222 provides an evaluation based on the respective category tag 250, such as referencing a particular portion of the reference database associated with the respective category tag (e.g., a first corpus 232-1 that includes a plurality of communications 240, each of which having the respective category tag 250 assigned to a respective communication 240 in the plurality of communications 240 of the first corpus 232-1). In some embodiments, the subset of category tags 250 includes at least 2 tags 250, at least 3 tags 250, at least 4 tags 250, at least 5 tags 250, at least 7 tags 250, at least 10 tags 250, at least 15 tags 250, at least 20 tags 250, or a combination thereof.

Block 452. Referring to block 452 of FIG. 4E, in some embodiments, the subset of category tags 250 includes a plurality of primary category tags 250. Additionally, each primary category tag 250 in the plurality of primary category tags 250 includes a corresponding plurality of secondary category tags 250 in the subset of category tags. Accordingly, in some embodiments, the assigning includes assigning a respective tag 250 in the secondary category tags 250 to the corresponding of information. For instance, in some embodiments, the plurality of primary category tags 250 includes an analyst report tag 250, an annual report tag 250, an asset acquisition tag 250, an asset sale tag 250, a clinical development update tag 250, a corporate update tag 250, a discard not relevant tag 250, a financing tag 250, an individual tag 250, a change in roles tag 250, a license agreement tag 250, a market research report tag 250, an entity merger tag 250, an entity acquisition tag 250, a new entity tag 250, an opinion tag 250, an option agreement tag 250, an other tag 250, a partnership tag 250, a preclinical update tag 250, a quarterly report tag, a regulatory report tag, a scientific analysis tag, a scientific publication tag, a patent publication tag, a future event tag, or a combination thereof. One of skill in the art will readily appreciate other types of tags 250 that are not expressly set forth by the present disclosure but are within the scope of the systems and methods described herein. Furthermore, in some embodiments, the secondary category tags 250 further include one or more corresponding tertiary category tags 250.

For instance, consider a respective primary category tag 250 of the financing tag 250. Accordingly, in some embodiments, the primary financing category tag 250 includes a plurality of secondary category tags 250 including a bridge loan tag 250, an announcement of a proposed public offering tag 250, a closing of an initial public offering tag 250, a closing of a public offering tag 250, a convertible note tag 250, a debt financing tag 250, an equity investment tag 250, a grant tag, a non-dilutive fund tag 250, an miscellaneous tag 250, a pipe tag 250, a pricing of an initial public offering tag 250, a pricing of a public offering tag 250, a private placement tag 250, a royalty investment tag 250, a seed funding tag 250, a series financing tag 250 (e.g., a series A tag 250, a series B tag, etc.), or a combination thereof. As another non-limiting example, consider a respective primary category tag 250 of the license agreement tag 250. Accordingly, in some embodiments, the primary license agreement tag 250 includes a plurality of secondary category tags 250 including a commercial license tag 250, an exclusive license tag 250, a patent license tag 250, a miscellaneous tag 250, or a combination thereof. However, the present disclosure is not limited thereto.

In some embodiments, the method 400 further extracts information from the first communication 240-1 based on one or more tags 250 assigned to the first communication 240-1. For instance, in some embodiments, the one or more tags 250 is associated with one or more corresponding heuristic instructions 224, such that if a respective tag 250 is assigned to the first communication 240-1, the classifier 222 further extracts information from the first communication 240-1 based on the heuristic instructions 224 associated with the respective tag 250. As a non-limiting example, consider the classification system 200 assigning the primary financing tag 250 to the first communication 240-1. Accordingly, in a determination of the assigning of the primary financing tag 250, the classifier extracts information from the communication 240 based on the primary financing tag 250, such as specific pricing information or financing information. In this way, in some embodiments, the method 400 extracts information that is specific to a primary category tag 250, such that an evaluation of the candidate subject is based on the information extracted from the first communication 240-1 through the assigning of the primary category tag 250, without having to extract information that is not related to the primary category tag 250.

Block 454. Referring to block 454 of FIG. 4E, the method 400 further includes applying a subset of tags 250 of the first plurality of tags 250 to the classifier 222 and the reference database 230. By applying the subset of tags 250, the method 400 obtains an evaluation of the candidate subject. Furthermore, by applying the subset of tags 250, as opposed to the first plurality of tags 250, the method 400 provides a more refined evaluation of the candidate subject by restricting the evaluation to those tags 250 of the subset of tags 250.

Block 456. Referring to block 456, in some embodiments, the subset of tags 250 is applied in response to a request to evaluate the candidate subject. For instance, in some embodiments, the candidate subject is associated with a first corpus of communications 232 that includes each communication 240 that is further associated with a first tag 250-1. In this way, a subset of tags 250 that includes the first tag 250 is applied in response to evaluate the first candidate subject. However, the present disclosure is not limited thereto.

Block 458. Referring to block 458, in some embodiments, the method 400 includes conducting the receiving of the first communication 240-1, the extracting of the information from the first communication 240-1, the assigning of one or more tags 250 to the extracted information from the first communication 240-1, and the applying a subset of the one or more tags 250 to obtain an evaluation of the first communication 240-1 for a second communication 240-2 in the plurality of communications (e.g., a second communication 240-2, a second communication of a corpus 232, etc.). In this way, the method 400 forms the subset of tags 250 of the first plurality of tags 250 based on an evaluation of the first plurality of tags 250 of the corresponding information of the first communication 240-1 with the second plurality of the corresponding information of the second communication 240-2.

Block 460, Referring to block 460 of FIG. 4F, in some embodiments, the evaluation formed by the applying includes a prediction of a future event, a prediction of a future communication 240 in the plurality of communications 232, a comparison of the candidate subject to a second candidate subject, or a combination thereof. For instance, in some embodiments, the evaluation is a validation of a candidate subject, an index associated with the candidate subject (e.g., an attractiveness index), a strategic position associated with the candidate subject (e.g., a position with respect to one or more competitors), an industry landscape, and the like. In some embodiments, the evaluation is an evaluation of a transaction, such as a corporate business transaction. In some embodiments, the evaluation is a diligence evaluation. In some embodiments, the evaluation is a valuation evaluation. In some embodiments, the evaluation is a document preparation evaluation. In some embodiments, the evaluation is a negotiation evaluation.

Said otherwise, in some embodiments, the systems (e.g., system 100 of FIG. 1) and methods (e.g., method 400 of FIGS. 4A through 4F) of the present disclosure provide an evaluation of a candidate subject based on an extraction of information from a first communication 240-1. The present disclosure extracts relevant information from the first communication 240-1 and then forms an evaluation based on this extracted information. In some embodiments, the information is extracted by comparing information in the first communication 240-1 with a plurality of predetermined information (e.g., a comparison with one or more communications 240 and/or tags 250 of the reference database 230). In some embodiments, the systems and methods of the present disclosure extract specific information (e.g., headers and/or tags 250) uniformly from a plurality of communications 240, allowing for a uniform dataset to be compiled (e.g., retained through the reference database 230). Moreover, by formatting the first communication, the systems and methods of the present disclosure provides a robust mechanism for providing an evaluation is a time efficient manner, such as immediately after publication of the first communication 240-1.

By providing an evaluation of the candidate subject through an extraction of information from the first communication 240-1 and applying one or more tags 250 to the extracted information of the first communication 240-1, the systems (e.g., system 100 of FIG. 1) and methods (e.g., method 400 of FIGS. 4A through 4F) of the present disclosure provide a classifier 222 that, in some embodiments, provides an understanding of patterns related to the candidate subject. In some embodiments, the classifier 222 extracts specific information from the first communication 240-1 and assign one or more tags to the extracted information. Furthermore, in some embodiments, the classifier 222 further extracts information based on the one or more tags assigned to the communication.

The foregoing descriptions of specific exemplary embodiments of the present invention have been presented for purposes of illustration and description. They are not intended to be exhaustive or to limit the invention to the precise forms disclosed, and obviously many modifications and variations are possible in light of the above teachings. The exemplary embodiments were chosen and described in order to explain certain principles of the invention and their practical application, to thereby enable others skilled in the art to make and utilize various exemplary embodiments of the present invention, as well as various alternatives and modifications thereof. It is intended that the scope of the invention be defined by the Claims appended hereto and their equivalents.

### REFERENCES CITED AND ALTERNATIVE EMBODIMENTS

All references cited herein are incorporated herein by reference in their entirety and for all purposes to the same extent as if each individual publication or patent or patent application was specifically and individually indicated to be incorporated by reference in its entirety for all purposes.

The present invention can be implemented as a computer program product that includes a computer program mechanism embedded in a non-transitory computer-readable storage medium. For instance, the computer program product could contain instructions for operating the user interfaces described with respect to FIGS. 2, 3, 5, 6, and 7. These program modules can be stored on a CD-ROM, DVD, magnetic disk storage product, USB key, or any other non-transitory computer readable data or program storage product.

Many modifications and variations of this invention can be made without departing from its spirit and scope, as will be apparent to those skilled in the art. The specific embodiments described herein are offered by way of example only. The embodiments were chosen and described in order to best explain the principles of the invention and its practical applications, to thereby enable others skilled in the art to best utilize the invention and various embodiments with various modifications as are suited to the particular use contemplated. The invention is to be limited only by the terms of the appended claims, along with the full scope of equivalents to which such claims are entitled.

What is claimed is:

1. A computer system for evaluating a candidate subject, the computer system comprising at least one processor, and a memory storing at least one program for execution by the at least one processor, the at least one program comprising instructions for:
(A) receiving, in electronic form, a first communication in a plurality of communications, wherein each communication in the plurality of communications comprises a respective plurality of text data, and wherein the first communication is associated with the candidate subject;
(B) extracting, using a trained classifier, a corresponding plurality of information from the respective text data of the first communication;
(C) assigning, using the trained classifier and a reference database, a tag to each respective information in a subset of information of the corresponding plurality of information, thereby collectively assigning a first plurality of tags in a set of tags to the corresponding plurality of information; and
(D) applying, to the trained classifier and the reference database, a subset of tags of the first plurality of tags, thereby obtaining an evaluation of the candidate subject.

2. The computer system of claim 1, wherein the candidate subject comprises an entity, a tangible asset, an intangible asset, or a combination thereof.

3. The computer system of claim 1, wherein the receiving (A) and/or the applying (D) is conducted in response to a request to evaluate the candidate subject.

4. The computer system of claim 1, wherein:
prior to the receiving (A), the at least one program further comprises instructions for polling for the first communication based on the association with the candidate subject, and
in accordance with a determination that the first communication exists, conducting the receiving (A).

5. The computer system of claim 1, wherein:
the reference database includes a corpus of communications, and
prior to the receiving (A), the at least one program further comprises instructions for training the trained classifier to evaluate the communication based on the corpus of communications.

6. The computer system of claim 5, wherein the corpus of communications is uniquely associated with the candidate subject.

7. The computer system of claim 5, wherein the at least one program further comprises instructions for adding the first communication to the corpus of communications.

8. The computer system of claim 1, wherein:
the text data of the first communication comprises unstructured text data, and
the receiving (A) further comprises parsing the unstructured text data for use with the trained classifier.

9. The computer system of claim 1, wherein the first communication is received from a first source, and, prior to the extracting (B), the at least one program further comprises instructions for validating the first source.

10. The computer system of claim 9, wherein the validating the first source comprises determining a type of source associated with the first source.

11. The computer system of claim 10, wherein, in accordance with a determination of the type of source associated with the first source, the validating the first source further comprises receiving a validation of the first source from a human subject.

12. The computer system of claim 10, wherein, in accordance with a determination of the type of source associated with the first source, the validating the first source further comprises assigning a weight of credibility to the first communication.

13. The computer system of claim 1, wherein the corresponding plurality of information of the extracting (B) contains a portion, less than all, of the text data.

14. The computer system of claim 1, wherein the trained classifier conducts the extracting (B) in accordance with a corresponding plurality of heuristic instructions that is associated with the extracting (B).

15. The computer system of claim 14, wherein the corresponding plurality of heuristic instructions comprises:
a first subset of heuristic instructions that extracts the first plurality of text data of the first communication into a first subset of information that contains a portion, less than all, of the corresponding plurality of information, and
a second subset of heuristic instructions that extracts a second plurality of text data of the second communication into a second subset of information that contains a portion, less than all, of the corresponding plurality of information.

16. The computer system of claim 15, wherein the at least one program further comprises instructions for:
conducting the extracting (B) in accordance with the first plurality of heuristic instructions and the assigning (C) based on the first subset of information, and
in accordance with a determination based on the assigning (C) of the first subset of information, conducting the extracting (B) in accordance with the second plurality of heuristic instructions and the assigning (C) based on the second subset of information.

17. The computer system of claim 1, wherein the at least one program further comprises instructions for:
conducting the receiving (A), the extracting (B), the assigning (C), and the applying (D) for a second communication in the plurality of communications, and
forming the subset of tags of the first plurality of tags based on an evaluation of the first plurality of tags of the corresponding information of the first communication with the second plurality of the corresponding information of the second communication.

18. The computer system of claim 1, wherein the evaluation formed by the applying (D) comprises a prediction of a future event, a prediction of a future communication in the plurality of communications, a comparison of the candidate subject to a second subject, or a combination thereof.

19. A method of evaluating a candidate subject at a computer system, the computer system comprising one or more processors, and memory coupled to the one or more processors, the memory comprising one or more programs configured to be executed by the one or more processors, the method comprising:
(A) receiving, in electronic form, a first communication in a plurality of communications, wherein each communication in the plurality of communications comprises a respective plurality of text data, and wherein the first communication is associated with the candidate subject;
(B) extracting, using a trained classifier, a corresponding plurality of information from the respective text data of the first communication;
(C) assigning, using the trained classifier and a reference database, a tag to each respective information in a subset of information of the corresponding plurality of information, thereby collectively assigning a first plurality of tags in a set of tags to the corresponding plurality of information; and
(D) applying, to the trained classifier and the reference database, a subset of tags of the first plurality of tags, thereby obtaining an evaluation of the candidate subject.

20. A non-transitory computer readable storage medium, wherein the non-transitory computer readable storage medium stores instructions, which when executed by a computer system, cause the computer system to perform a method comprising:
(A) receiving, in electronic form, a first communication in a plurality of communications, wherein each communication in the plurality of communications comprises a respective plurality of text data, and wherein the first communication is associated with the candidate subject;
(B) extracting, using a trained classifier, a corresponding plurality of information from the respective text data of the first communication;
(C) assigning, using the trained classifier and a reference database, a tag to each respective information in a subset of information of the corresponding plurality of information, thereby collectively assigning a first plurality of tags in a set of tags to the corresponding plurality of information; and
(D) applying, to the trained classifier and the reference database, a subset of tags of the first plurality of tags, thereby obtaining an evaluation of the candidate subject.
